(19) United States
(12) Patent Application Publication
(10) Pub. No.: US 2026/0034147 A1
(43) Pub. Date: Feb. 5, 2026

(54) COMPOUNDS AND THEIR USES FOR ALLEVIATING MENOPAUSE-ASSOCIATED SYMPTOMS

(71) Applicant: ESTETRA SRL, Liège (BE)

(72) Inventors: Melanie Taziaux, Liège (BE); Glwadys Rausin, Trooz (BE); Maud Jost, Liège (BE); Marie Mawet, Angleur (BE)

(73) Assignee: ESTETRA SRL, Liège (BE)

(21) Appl. No.: 19/355,373

(22) Filed: Oct. 10, 2025

Related U.S. Application Data
(63) Continuation of application No. 17/972,403, filed on Oct. 24, 2022, now Pat. No. 12,458,651.
Application No. 17/972,403 is a continuation of application No. 17/963,712, filed on Oct. 11, 2022, now Pat. No. 11,666,585.
Application No. 17/963,712 is a continuation of application No. 17/893,999, filed on Aug. 23, 2022, now Pat. No. 12,390,478.
Application No. 17/893,999 is a continuation of application No. 17/048,538, now Pat. No. 11,452,733.

(30) Foreign Application Priority Data
Apr. 19, 2018 (EP) 18168336.8
May 30, 2018 (EP) 18174982.1
Jan. 4, 2019 (EP) 19150423.2

(51) Int. Cl. A61K 31/585; A61K 9/00; A61K 31/55; A61K 31/565; A61K 31/57; A61K 31/573; A61P 15/12

(52) U.S. Cl. CPC A61K 31/585; A61K 9/0053; A61K 31/55; A61K 31/565; A61K 31/57; A61K 31/573; A61P 15/12

(57) ABSTRACT

The present invention relates to a hormone replacement therapy, to the associated compounds and to the associated packaging units, for alleviating menopause-associated symptoms which is based on the administration to a female mammal of an estetrol component at specified daily doses, optionally in combination with a progestogenic component.

The therapy enjoys a statistically significant efficacy combined with a favourable profile for side effects compared to currently available methods for alleviating menopause-associated symptoms.

### FIELD OF THE INVENTION

The present invention relates to hormone replacement therapy, to the associated compounds and to the associated packaging units, for alleviating menopause-associated symptoms which is based on the administration to a female mammal of an estetrol component at specified daily doses, optionally in combination with a progestogenic component.

As further detailed herein, the therapy displays statistically significant efficacy combined with a favourable profile for side effects compared to currently available methods for alleviating menopause-associated symptoms.

### BACKGROUND ART

Hormone replacement therapy (HRT) is used to describe either unopposed oestrogen use (for women who have undergone hysterectomy) or combined oestrogen-progestin therapy (for women still having a uterus). The goal of HRT is to relieve menopausal symptoms, most importantly vasomotor symptoms (VMS), such as hot flushes. Other diseases and symptoms associated with perimenopause and menopause that respond to oestrogen therapy include osteoporosis, vaginal atrophy, and sleep disturbances (when related to hot flushes).

VMS occur most often in the late menopausal transition and early post-menopause. VMS are the most significant menopausal complaints. Estimates suggest that about 75% of women who are more than 50 years old will suffer from hot flushes (Utian et al., 2005, “Comparative controlled trial of a novel oral estrogen therapy, estradiol acetate, for relief of menopause symptoms” Menopause 12(6): 708-715). Most experience hot flushes for about two years, although around 10% suffer for more than 10 years (Rodstrom et al., 2002, “A longitudinal study of the treatment of hot flushes: the population study of women in Gothenburg during a quarter of a century” Menopause 9(3): 156-161). VMS can contribute towards physical and psycho-social impairment, with a consequent reduction in quality of life, and are one of the main reasons why women may seek medical care for the menopause (Santoro, 2008, “Symptoms of menopause: hot flushes” Clin Obstet Gynecol 51(3): 539-548).

The epithelial linings of the vagina and urethra are very sensitive to oestrogen, and oestrogen deficiency leads to thinning both epithelia. This results in vulvovaginal atrophy (VVA) and urinary complaints, causing symptoms of vaginal dryness, itching, dyspareunia, dysuria, urinary frequency and an increased risk of recurrent urinary infections. In early 2014, the International Society for the Study of Women's Sexual Health (ISSWSH) and the North American Menopause

Society (NAMS) endorsed the new terminology “genitourinary syndrome of menopause (GSM)” to replace the VVA terminology. The rationale for using this new terminology was that VVA term was too restrictive whereas GSM was a more comprehensive term that includes symptomatic VVA as well as lower urinary tract symptoms related to low oestrogen levels (Portman et al., 2014, “Genitourinary syndrome of menopause: new terminology for vulvovaginal atrophy from the International Society for the Study of Women's Sexual Health and the North American Menopause Society” Menopause 21(10): 1063-1068). Note that the GSM terminology has not yet been adopted in guidance for industry issued by the FDA and EMA, therefore the VVA terminology will be used herein.

Oestrogen therapy remains the gold standard for relief of menopausal symptoms, in particular VMS. All routes of administration appear to be equally effective for symptom relief, but their metabolic effects differ. Oestrogens should be administered continuously; past regimens where oestrogen was administered day 1 to day 25 of the calendar month are considered to be obsolete. Women will often get VMS during the days off, and there is no known advantage to stopping for several days each month.

With current HRT, all women with an intact uterus need a progestin in addition to oestrogen to prevent endometrial hyperplasia—and subsequent carcinoma—which occurs after as little as 6 months of oestrogen therapy. The progestin may be administered continuously or sequentially (e.g., 10 to 14 days each month or for 14 days every 3 months).

Oestrogen therapy is the most consistently effective treatment used in the US and Europe for menopausal VMS. Following the safety issues reported in the primary Women's Health Initiative publications (Anderson et al., 2004, “Effects of conjugated equine estrogen in postmenopausal women with hysterectomy: the Women's Health Initiative randomized controlled trial” JAMA 291(14): 1701-1712) and with continued subject requests for treatment, a challenge to clinicians has been to identify the lowest effective dose of oestrogen for alleviating menopausal symptoms (Simon et al., 2007, “Menopausal hormone therapy for vasomotor symptoms: balancing the risksand benefits with ultra-low doses of estrogen” Expert Opin Investig Drugs 16(12): 2005-2020). In addition, it is a challenge to develop a safer oestrogen than those currently used.

In a 2016 publication, Coelingh Bennink et al; (“Clinical effects of the fetal estrogen estetrol in a multiple-rising-dose study in postmenopausal women”, Maturitas, 2016 September; 91:93-100) report a partly randomized, open-label, multiple-rising-dose study in 49 postmenopausal women who received either 2 mg estetrol or 2 mg estradiol-valerate per day for 28 days (randomized part of the study) followed by dose-escalation at 10, 20 and 40 mg estetrol per day (non-randomized part of the study). The authors compared the efficacy of estradiol-valerate (2 mg) and only two doses of estetrol (2 and 10 mg) on the relief of hot flushes on subjects with >35 hot flushes per week at screening (note a. to Table 1). The authors report a decrease in the mean number of hot flushes and sweating already with the 2 mg estetrol per day dose, while at the same time endometrial thickness was found to be stable in the 2 mg estetrol group but increasing in the 10 mg estetrol group (abstract).

From this study, since the authors did not measure VMS in the 20 mg and 40 mg estetrol groups, it is apparent to the skilled person that the authors considered only the lower doses (2 mg and 10 mg) to be suitable for the management of VMS. Further, the skilled person learns that a daily dose of 2 mg estetrol is as effective as a daily dose of 10 mg estetrol at decreasing the number of hot flushes per day (see for example FIG. 3 in Coelingh Bennink et al., Maturitas, 2016). In addition, the publication teaches the skilled person that the 2 mg daily dose does not alter endometrial thickness while the 10 mg daily dose has an important effect on endometrial thickness (see for example FIG. 2 in Coelingh Bennink et al., 2016). The skilled person would thus conclude that the Minimal Effective Dose of estetrol for the treatment of VMS is 2 mg per day.

### SUMMARY OF THE INVENTION

Against this background, the present inventors have now surprisingly found that the Minimal Effective Dose of estetrol for the alleviation of VMS in perimenopausal and postmenopausal women is of about 15 mg daily.

The determination of the Minimal Effective Dose indeed revolves around a number of parameters which take into account not only the mean number of VMS per day (as was done by Coelingh Bennink et al in Maturitas, 2016), but also the severity of VMS, and also a weekly weighted score, as further defined herein.

Thus, in terms of efficacy, it was surprisingly found that a daily dose of 15 mg was required to obtain a definite effect on VMS.

Furthermore, it was observed by the present inventors that several side effects of the treatment, as reflected for example in the number of treatment-emergent adverse events (TEAEs), or in the number of patients leaving the study in each group, or in the number of biopsies which had to be performed in each group, tend to reach a plateau at around 10 mg per day, and do not worsen when the dose is further increased. Surprisingly, some of these parameters in fact regress from 10 mg to 15 mg daily, thus indicating that the adverse events could be more bearable when the dose is higher than 10 mg per day.

Based on this surprising finding, and on measurements performed when a 20 mg and a 30 mg daily doses are administered, the present inventors have thus come to the unexpected conclusion that an optimal daily dose of estetrol for alleviation of VMS in postmenopausal women is found in the range of 15 mg to 25 mg. This range of doses indeed allows statistically significant effects on VMS to be observed, as is shown in the examples, while maintaining the treatment side effects within an acceptable window.

In the following numbered paragraphs 1 to 14, embodiments of the invention are described.

1. A composition for use in alleviating menopause-associated symptoms, wherein said composition comprises an estetrol component and wherein said composition is administered at a daily amount equivalent to from about 15 mg to about 25 mg of estetrol.

2. A composition for use in alleviating vasomotor symptoms selected from hot flashes, sweating attacks, night sweats, chills, increased perspiration, palpitations and combinations thereof, wherein said composition comprises an estetrol component and wherein said composition is administered at a daily amount equivalent to from about 15 mg to about 25 mg of estetrol.

3. A composition for use in alleviating emotional aspects of the menopausal transition selected from depression, irritability, mood changes, insomnia, sleep disturbance, anxiety, nervous tension and combinations thereof, wherein said composition comprises an estetrol component and wherein said composition is administered at a daily amount equivalent to from about 15 mg to about 25 mg of estetrol.

4. A composition for use in alleviating physiological aspects of the menopausal transition selected from joint pain, loss of bone density, urinary tract infections, urinary incontinence, dryness of the vagina, uterine prolapse, changes in skin texture, weight gain, dyspareunia, cardiovascular diseases, diabetes and combinations thereof, wherein said composition comprises an estetrol component and wherein said composition is administered at a daily amount equivalent to from about 15 mg to about 25 mg of estetrol.

5. A composition for use in reducing VMS frequency, VMS severity, hot flush weekly weighted score, dryness of the vagina, dyspareunia and combinations thereof, or for use in improving Quality of Life according to the MRS and/or the MENQOL questionnaires, wherein said composition comprises an estetrol component and wherein said composition is administered at a daily amount equivalent to from about 15 mg to about 25 mg of estetrol.

6. The composition for use according to any one of paragraphs 1-5 above wherein the composition is administered daily for at least 1, at least 2, at least 4, at least 6, at least 12 weeks.

7. The composition for use of any one of paragraphs 1-6 wherein a second composition comprising a progestogenic component is additionally used.

8. The composition for use of paragraph 7 wherein said progestogenic component is selected from drospirenone, progesterone and dydrogesterone.

9. The composition for use of paragraph 7 wherein progesterone is administered at a daily dose of from 50 mg to 200 mg.

10. The composition for use of any one of paragraphs 1-6 wherein a second composition comprising bazedoxifene is additionally used.

11. A composition for use according to any one of paragraphs 7-10 wherein said second composition comprising a progestogenic component or bazedoxifene is the same as the composition comprising the estetrol component.

12. A composition for use according to any one of paragraphs 1-11 wherein the estetrol component is estetrol, preferably estetrol monohydrate.

13. A composition for use according to any of the preceding paragraphs wherein the composition is formulated as an oral dosage unit.

14. The composition for use according to paragraph 13 wherein the oral dosage unit is formulated to correspond to a daily dosage unit.

In the following numbered paragraphs 15 to 28, additional embodiments of the invention are described.

15. A method of alleviating menopause-associated symptoms which comprises administration of a composition comprising an estetrol component at a daily amount equivalent to from about 15 mg to about 25 mg of estetrol.

16. A method of alleviating vasomotor symptoms selected from hot flashes, sweating attacks, night sweats, chills, increased perspiration, palpitations and combinations thereof, which comprises administration of a composition comprising an estetrol component at a daily amount equivalent to from about 15 mg to about 25 mg of estetrol.

17. A method of alleviating emotional aspects of the menopausal transition selected from depression, irritability, mood changes, insomnia, sleep disturbance, anxiety, nervous tension and combinations thereof, which comprises administration of a composition comprising an estetrol component at a daily amount equivalent to from about 15 mg to about 25 mg of estetrol.

18. A method of alleviating physiological aspects of the menopausal transition selected from joint pain, loss of bone density, urinary tract infections, urinary incontinence, dryness of the vagina, uterine prolapse, changes in skin texture, weight gain, dyspareunia, cardiovascular diseases, diabetes and combinations thereof, which comprises administration of a composition comprising an estetrol component at a daily amount equivalent to from about 15 mg to about 25 mg of estetrol.

19. A method of reducing VMS frequency, VMS severity, hot flush weekly weighted score, dryness of the vagina, dyspareunia and combinations thereof, or of improving Quality of Life according to the MRS and/or the MENQOL questionnaires, which comprises administration of a composition comprising an estetrol component at a daily amount equivalent to from about 15 mg to about 25 mg of estetrol.

20. The method according to any one of paragraphs 15-19 above wherein the composition is administered daily for at least 1, at least 2, at least 4, at least 6, at least 12 weeks.

21. The method according to any one of paragraphs 15-20 wherein a second composition comprising a progestogenic component is additionally administered.

22. The method according to paragraph 21 wherein said progestogenic component is selected from drospirenone, progesterone and dydrogesterone.

23. The method according to paragraph 22 wherein progesterone is administered at a daily dose of from 50 mg to 200 mg.

24. The method according to any one of paragraphs 15-20 wherein a second composition comprising bazedoxifene is additionally administered.

25. The method according to any one of paragraphs 21-24 wherein said second composition comprising a progestogenic component or bazedoxifene is the same as the composition comprising the estetrol component.

26. The method according to any one of paragraphs 15-25 wherein the estetrol component is estetrol, preferably estetrol monohydrate.

27. The method according to any of the preceding paragraphs wherein the composition is formulated as an oral dosage unit.

28. The method according to paragraph 27 wherein the oral dosage unit is formulated to correspond to a daily dosage unit.

In the following numbered paragraphs 29 to 42, additional embodiments of the invention are described.

29. Use of an effective amount of an estetrol component in the manufacture of a composition for alleviating menopause-associated symptoms, wherein the estetrol component is used at a daily amount equivalent to from about 15 mg to about 25 mg of estetrol.

30. Use of an effective amount of an estetrol component in the manufacture of a composition for alleviating vasomotor symptoms selected from hot flashes, sweating attacks, night sweats, chills, increased perspiration, palpitations and combinations thereof, wherein the estetrol component is used at a daily amount equivalent to from about 15 mg to about 25 mg of estetrol.

31. Use of an effective amount of an estetrol component in the manufacture of a composition for alleviating emotional aspects of the menopausal transition selected from depression, irritability, mood changes, insomnia, sleep disturbance, anxiety, nervous tension and combinations thereof, wherein the estetrol component is used at a daily amount equivalent to from about 15 mg to about 25 mg of estetrol.

32. Use of an effective amount of an estetrol component in the manufacture of a composition for alleviating physiological aspects of the menopausal transition selected from joint pain, loss of bone density, urinary tract infections, urinary incontinence, dryness of the vagina, uterine prolapse, changes in skin texture, weight gain, dyspareunia, cardiovascular diseases, diabetes and combinations thereof, wherein the estetrol component is used at a daily amount equivalent to from about 15 mg to about 25 mg of estetrol.

33. Use of an effective amount of an estetrol component in the manufacture of a composition for reducing VMS frequency, VMS severity, hot flush weekly weighted score, dryness of the vagina, dyspareunia and combinations thereof, or for improving Quality of Life according to the MRS and/or the MENQOL questionnaires, wherein the estetrol component is used at a daily amount equivalent to from about 15 mg to about 25 mg of estetrol.

34. The use according to any one of paragraphs 29-33 above wherein the composition is administered daily for at least 1, at least 2, at least 4, at least 6, at least 12 weeks.

35. The use according to any one of paragraphs 29-34 wherein a second composition comprising a progestogenic component is additionally administered.

36. The use according to paragraph 35 wherein said progestogenic component is selected from drospirenone, progesterone and dydrogesterone.

37. The use according to paragraph 35 wherein progesterone is administered at a daily dose of from 50 mg to 200 mg.

38. The use according to any one of paragraphs 29-34 wherein a second composition comprising bazedoxifene is additionally administered.

39. The use according to any one of paragraphs 35-38 wherein said second composition comprising a progestogenic component or bazedoxifene is the same as the composition comprising the estetrol component.

40. The use according to any one of paragraphs 29-39 wherein the estetrol component is estetrol, preferably estetrol monohydrate.

41. The use according to any of the preceding paragraphs wherein the composition is formulated as an oral dosage unit.

42. The use according to paragraph 41 wherein the oral dosage unit is formulated to correspond to a daily dosage unit.

In the following numbered paragraphs 43 to 50, additional embodiments of the invention are described.

43. A packaging unit comprising at least 14, preferably at least 21, even more preferably at least 28, containers for holding separately packaged and individually removable daily solid oral dosage forms, wherein each container comprises at least one daily solid oral dosage form comprising from about 15 mg to about 25 mg of estetrol.

44. The packaging unit according to paragraph 43, wherein the packaging unit additionally comprises at least 10, preferably 12, more preferably 14, additional containers for holding separately packaged and individually removable daily, preferably solid, oral dosage forms, wherein each additional container comprises at least one daily, preferably solid, oral dosage form comprising a progestogenic component.

45. The packaging unit according to paragraph 44, wherein each of the additional containers for holding the dosage forms comprising the progestogenic component are individually visually arranged next to a container holding a solid dosage form comprising estetrol when these two solid dosage forms have to be administered on the same day.

46. The packaging unit according to paragraphs 44 or 45, wherein said progestogenic component is selected from drospirenone, progesterone and dydrogesterone.

47. The packaging unit according to paragraph 46, wherein said progestogenic component is progesterone and wherein each said additional containers comprises at least one daily oral dosage form comprising about 200 mg of progesterone.

48. The packaging unit according to paragraph 43, wherein the packaging unit additionally comprises the same number of additional containers for holding separately packaged and individually removable daily, preferably solid, oral dosage forms, wherein each additional container comprises at least one daily, preferably solid, oral dosage form comprising a progestogenic component, preferably wherein said progestogenic component is selected from drospirenone, progesterone and dydrogesterone, even more preferably wherein said progestogenic component is progesterone and wherein said additional container comprises at least one daily oral dosage form comprising about 100 mg of progesterone.

49. The packaging unit according to paragraph 43, wherein at least 10, preferably 12, more preferably 14, of said daily solid oral dosage forms additionally comprises a progestogenic component.

50. The packaging unit according to paragraph 43, wherein each said daily solid oral dosage form additionally comprises a progestogenic component.

The skilled person will understand that the embodiments described in numbered paragraphs 44 to 50 may equivalently be presented as a kit-of-parts containing a first packaging unit, e.g. a blister pack, containing the daily oral dosage units comprising the estetrol component, and a second, distinct, packaging unit, e.g. a second, distinct, blister pack, containing the daily oral dosage units comprising the progestogenic component.

The skilled person will additionally know that, within the scope of the present invention, each packaging unit, e.g. blister pack, may be numbered or otherwise marked.

Within the scope of the invention, each packaging unit may be a sealed blister pack with a cardboard, paperboard, foil plastic backing and enclosed in a suitable cover.

In a particular embodiment of the invention the packaging unit comprises 28 containers or a multiple of 28 containers, such as 2 to 12 multiple of 28 containers.

### DETAILED DESCRIPTION OF THE INVENTION

### Definitions

The term “estetrol component”, as used throughout this document, encompasses substances selected from the group consisting of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms; and combinations thereof. Even more preferably, the estetrol component is estetrol (including estetrol hydrates). Most preferably, the estetrol component contained in the dosage unit is estetrol monohydrate.

The term “estetrol” as used herein refers to 1,3,5 (10)-estratrien-3,15alpha, 16alpha, 17beta-tetrol or 15alpha-hydroxyestriol as well as hydrates of estetrol, e.g. estetrol monohydrate.

The term “progestogenic component” is defined as a substance that is capable of triggering a progestogenic response in vivo or a precursor which is capable of liberating such a substance in vivo. Usually progestogenic components are capable of binding to a progestogen receptor.

In the context of the present invention, other compounds may be used in conjunction with the estetrol component for administering to women who have a uterus. Selective Estrogen Receptor Modulators (SERMs) defines a category of such compounds, which are contemplated as useful complements to the estetrol component in the methods of the invention. A preferred SERM for use in the context of the present invention is bazedoxifene.

In the methods and compositions further described herein, it has to be understood that when reference is made to a “progestogenic component”, such reference includes SERMs and in particular bazedoxifene.

“About” as used herein referring to a measurable value such as a parameter, an amount, a temporal duration, and the like, is meant to encompass variations of +/−10% or less, more preferably +/−5% or less, even more preferably +/−1% or less of and from the specified value, in so far such variations are appropriate to perform in the disclosed invention. However, it is to be understood that the value to which the modifier “about” refers is itself also specifically disclosed.

The term “an effective amount” refers to an amount necessary to obtain a physiological effect. The physiological effect may be achieved by one dose or by repeated doses.

The term “perimenopause” as used herein (also in the context of “perimenopausal women” and “perimenopausal subject” and the like) refers to a period of life which begins approximately three to four years prior to menopause and ends one year after the final menstrual period. Perimenopause is characterized by persistent irregular menstrual cycles, extreme fluctuations in hormonal levels, frequent anovulation and the appearance of VMS (for reference: Harlow et al., Menopause, Vol. 19, No. 4, 2012, “Executive summary of the Stages of Reproductive Aging Workshop +10: addressing the unfinished agenda of staging reproductive aging”—see in particular FIG. 2 therein). During this transition period, the emphasis of clinical care changes. Although women still need effective contraception during perimenopause, issues including loss of bone mineral density, menstrual cycle changes, and vasomotor instability also need to be addressed. A significant number of women also experience depressive symptoms, such as mood swings, irritability, and poor concentration (Bosworth et al., Psychosom Med., 2001 July-August;63(4):603-8, “Depressive symptoms, menopausal status, and climacteric symptoms in women at midlife”).

The term “postmenopause” as used herein (also in the context of “postmenopausal women” and “postmenopausal subject” and the like) covers firstly spontaneous post-menopausal women, i.e. women who have encountered natural menopause defined as the permanent cessation of menstrual periods, determined retrospectively after a woman has experienced 12 months of amenorrhea without any other obvious pathological or physiological cause. It occurs at a median age of 51.4 years in normal women and is a reflection of complete, or near complete, ovarian follicular depletion, with resulting hypoestrogenemia (with estradiol levels often below 20 pg/mL) and high follicle-stimulating hormone (FSH level >40 IU/L) concentrations. The term “postmenopause” also includes menopause as the consequence of premature ovarian failure, surgery (ovariectomy for example), chemotherapy or radiotherapy for cancer, and certain diseases (for example, infections or hypothyroidism).

The term “VMS” as used herein (alternatively in its non-abbreviated form, “vasomotor symptoms”) corresponds to thermo-regulatory disturbances characteristic of menopause. VMS includes hot flashes (also sometimes spelled “hot flushes”), sweating attacks such as night sweats, chills and increased perspiration, and palpitations. VMS are episodes of profuse heat accompanied by sweating and flushing, experienced predominantly around the head, neck, chest, and upper back. As further defined below, VMS are classified into mild, moderate and severe categories.

The terms “Menopause-associated symptoms”, as used herein, is used to describe VMS as defined above, but also includes the emotional aspects of the menopausal transition (including, but not limited to, depression, irritability, mood changes, insomnia, sleep disturbance, anxiety and nervous tension) and the physiological aspects of the menopausal transition (including, but not limited to joint pain, loss of bone density, urinary tract infections, urinary incontinence, dryness of the vagina, uterine prolapse, changes in skin texture, weight gain and dyspareunia, as well as cardiovascular diseases and diabetes).

As used herein, the terms “Quality of Life” (and the abbreviation “QoL”) refers to a parameter which can be assessed, for example, by using questionnaires, such as, by way of example and not limitation, the “Menopause Rating Scale” questionnaire (Heinemann et al., 2003, “International versions of the Menopause Rating Scale (MRS)” Health Qual Life Outcomes 1:28; Heinemann et al., 2004, “The Menopause Rating Scale (MRS) scale: A methodological review”. Health Qual Life Outcomes 2:45; Heinemann et al., 2004, “The Menopause Rating Scale (MRS) asoutcome measure for hormone treatment? A validation study”. Health Qual Life Outcomes 2:67; as further detailed below in Example 1 Section C.) or the MENQOL questionnaire (The Menopause-specific Quality of Life (MENQOL) questionnaire, Hilditch et al.; Maturitas 1996; A menopause-specific quality of life questionnaire: development and psychometric properties; 24(3); p.161-175).

As used herein, “BMI” (or “Body Mass Index” in its non-abbreviated form refers to an index relating to weight and height of a subject, which is calculated by dividing the subject's weight in kilograms by the subject's height in meters squared. A BMI of 27.3 or more classifies the female subject as “overweight” while a BMI of 30 or more classifies the subject as “obese”.

### Determination of the Minimum Effective Dose

To determine the Minimum Effective Dose (MED), firstly the selection criteria required that patients presented at least 7 moderate to severe VMS/day or at least 50 moderate to severe VMS/week in the week preceding randomization. Secondly, a placebo group was included in a double-blind fashion. Thirdly, the total patient number was such that statistical significance could be obtained between separate arms of the study. Fourthly, the absolute change from baseline in weekly frequency of moderate to severe VMS was taken into account. An analysis of covariance (ANCOVA) was performed based on the untransformed change in weekly frequency of moderate to severe VMS from baseline. Finally, the change in severity from baseline was evaluated. For this, the Severity Scoring System of VMS was documented by the subjects as follows:
a score of Mild (1) for a sensation of heat without sweating;
a score of Moderate (2) for a sensation of heat with sweating/the subject is able to continue activity; and
a score of Severe (3) for a sensation of heat with sweating/which causes cessation of activity.

In addition, a severity score of zero was attributed to the patients who have experienced a 100% VMS relief during a given week.

From these score records, the severity at baseline was calculated by taking into account only moderate and severe VMS, such that the total number of moderate VMS during the 7 days of the baseline week was multiplied by 2 and added to the total number of severe VMS during the 7 days of the baseline week multiplied by 3. This total was then divided by the total number of moderate and severe VMS during the baseline week.

The severity at weeks 4 and 12 was calculated using the following formula for each of these weeks:
the number of mild VMS during 7 days multiplied by 1;
the number of moderate VMS during 7 days multiplied by 2;
the number of severe VMS during 7 days multiplied by 3.
   and adding the 3 resulting numbers together before dividing this total by the total number of mild, moderate and severe VMS during the 7 days of the week.

This method was used to generate the data included in Example 1 further below, under section A. b) “VMS Severity”.

In an alternative embodiment, the VMS Severity is assessed as follows: the severity at baseline is also calculated by taking into account only moderate and severe VMS, such that the total number of moderate VMS during the 7 days of the baseline week is multiplied by 2 and added to the total number of severe VMS during the 7 days of the baseline week multiplied by 3. This total is then divided by the total number of moderate and severe VMS during the baseline week.

The severity at weeks 4 and 12, however, is calculated using the following formula for each of these weeks
the number of moderate VMS during 7 days multiplied by 2;
the number of severe VMS during 7 days multiplied by 3.
   and adding the 2 resulting numbers together before dividing this total by the total number of moderate and severe VMS during the 7 days of the week.

In another embodiment, the severity score can be calculated according to the method described in Archer et al, 2014 (A randomized, double-blind, placebo-controlled study of the lowest effective dose of drospirenone with 17A-estradiol for moderate to severe vasomotor symptoms in postmenopausal women; Menopause, 2014; 21(3); p.227-235). According to this method, a daily severity score is calculated using the following formula for each day during 7 days
the number of moderate VMS during 1 day is multiplied by 2;
the number of severe VMS during 1 day is multiplied by 3;
the 2 resulting numbers are added together;
the addition result is in turn divided by the total number of VMS during the same day (moderate and severe).

The same formula is applied during 7 consecutive days, the 7 resulting numbers are summed together and divided by 7 to calculate the “weekly mean daily severity” score of moderate to severe VMS.

Further, the terms “Hot Flush Weekly Weighted Score”, as used herein, corresponds to a score taking into account frequency and severity which is calculated by using the severity score (as calculated above): [(1×No. of mild VMS)+(2×No. of moderate VMS)+(3×No. of severe VMS)] during a 7 day period.

Such a weighted score was for example used by Notelovitz et al. (“Initial 17β-Estradiol Dose for Treating Vasomotor Symptoms”; Obstetrics and Gynaecology, Vol. 95(5), May 2000, p. 726-731). This score was used to generate the data included in Example 1 further below, under section A. c) “Hot Flush Weekly Weighted Score”.

In alternative embodiments, any combination of the above described methods maybe employed to evaluate the weekly severity and/or frequency of VMS.

### Hormone Replacement Therapy

The present therapy usually employs continuous administration of the estetrol component during a period of at least 10 days, preferably of at least 20 days.

The estetrol component is administered at a daily dose of from about 15 mg to about 25 mg.

In a specific embodiment, the estetrol component is administered at a daily dose of from about 15 mg to less than 20 mg.

In another specific embodiment, the estetrol component is administered at a daily dose of from more than 20 mg to about 25 mg.

In one embodiment, the present therapy is administered to non-hysterectomied patients. In a particular embodiment, the present therapy involves daily administration of about 20 mg of the estetrol component, preferably to non-hysterectomied patients.

In the cases when the present therapy is administered to a patient who has undergone hysterectomy, the estetrol component is preferably administered as sole active ingredient.

When the present therapy is administered to non-hysterectomied patients, the estetrol component may be administered as sole active ingredient or may be administered together with an optional progestogenic component. Said optional progestogenic component may be administered continuously (i.e. every day in addition to the estetrol component) or sequentially (wherein sequentially means an administration of the progestogenic component during, for example, 10 to 14 days each month or during 14 days every 3 months).

The terms “continuous” /“continuously” as used herein, means that the components are administered at relatively regular intervals, with no (therapeutically) significant interruptions. Naturally, minor interruptions may occur that do not affect the overall effectiveness of the present method, and indeed such aberrations are encompassed by the present invention. In a preferred embodiment, and more arithmetically, the administration regimen is deemed to be continuous if the longest interval between 2 subsequent administrations is not more than 3.5 times as long as the average interval. Even more preferably said longest interval is not more than 2.5 times, most preferably not more than 1.5 times as long as the average interval.

In one embodiment, the optional progestogenic component is administered via a non-oral route, for example using an Intra Uterine Device (IUD). In one embodiment said IUD delivers the progestogenic component levonorgestrel. In one such embodiment, the IUD is the Mirena® IUD or the Levosert® IUD.

In one embodiment, the present therapy employs oral, sublingual, buccal, or sublabial administration of the estetrol component. These latter 3 modes of administration offer the advantages that the estetrol component does not have to pass through the digestive system and avoids first-pass liver exposure. Furthermore, these modes of administration provide a rapid onset of action.

The term “sublingual” as used herein refers to the pharmacological route of administration by which the estetrol component diffuses into the blood through tissues under the tongue.

The term “buccal” as used herein refers to the pharmacological route of administration by which the estetrol component diffuses into the blood through tissues of the buccal vestibule, the area inside the mouth between the lining of cheek (the buccal mucosa) and the teeth/gums.

The term “sublabial” as used herein refers to the pharmacological route of administration by which the estetrol component is placed between the lip and the gingiva.

In the present method, the estetrol and progestogenic components may be administered in separate dosage units. However, it is also possible and indeed very convenient to combine these two components into a single dosage unit.

In the method according to the present invention the combination of the progestogenic and estetrol component is suitably administered continuously during a period of at least 10 days.

The invention may suitably be reduced to practice in the form of a variety of administration methods that are known to the person skilled in the art. Amongst these methods are the methods making use of monophasic preparations, which contain dosage units with a constant amount of the estetrol component and of the optional progestogenic component.

In the embodiment of the invention where sequential administration of the progestogenic component is chosen, it is also possible and convenient to combine the components into a single dosage unit for the days when the two components are administered.

In one embodiment of the invention, the hormone replacement therapy is administered to a perimenopausal subject. In this embodiment, the subject will benefit from the alleviation of VMS through the administration of the composition of the invention, while simultaneously benefiting from a contraceptive effect. In the specific perimenopausal population, contraception is indeed often required, and since VMS appear during this time of life, the treatment of the invention will uniquely address these two needs in a single and simple treatment.

In another embodiment of the invention, the hormone replacement therapy is administered to a postmenopausal subject.

In a particular embodiment of the invention, the hormone replacement therapy is beneficially administered to take advantage of the absence of effect of the smoking status of the subject on the therapy of the invention. It has indeed long been known that smoking significantly reduces serum estrogen concentrations, such as reported for example in the clinical study analysis of Bjarnason et al. (Bjarnason et al.; Climacteric 2012; Acute and long-term estradiol kinetics insmoking postmenopausal women; 15:5; p.449-454) who found that in the estrogen group, smoking leads to significantly lower levels of both serum estrone and serum estradiol at all post-randomization time points, while no differences between smokers and non-smokers were seen on placebo. Bjarnason et al. conclude that smoking reduces serum estrogens at both trough and after 2 h in postmenopausal women on estrogen treatment, that the effect of smoking on estrogen concentrations is fully expressed in women smoking ten or less cigarettes daily, and that the influence of smoking upon the metabolism of estrogen therapy is constant and without dose-response for standard smoking intensities.

In this particular embodiment, based on the surprising finding that the therapy of the invention is not affected by the smoking status of the subject, the hormone replacement therapy is preferably administered to a patient population smoking 5 or more cigarettes daily, to a patient population smoking 10 or more cigarettes daily, or to a patient population smoking 15 or more cigarettes daily.

In yet another particular embodiment of the invention, the hormone replacement therapy is beneficially administered to take advantage of the absence of effect of the BMI of the subject on the therapy of the invention. It has indeed been surprisingly found that contrary to the prior art hormone replacement therapies, the efficacy of the therapy of the invention is not affected by the

BMI value of the subject. In this particular embodiment, the hormone therapy of the invention is preferably administered to a subject whose BMI is 25 or more, 28 or more, 30 or more, 33 or more, 35 or more, 37 or more, or 40 or more, In this particular embodiment, the hormone therapy of the invention is preferably administered to an overweight subject, or to an obese subject.

The hormone replacement therapy of the invention was found to be particularly beneficial for alleviating menopause-associated symptoms while having an extremely limited impact on a large number of hepatic, haemostatic, endocrine and metabolic parameters. It is indeed the case that the HRT treatments of the prior art have a negative impact on these parameters. Surprisingly and very beneficially, the treatment of the present invention was found by contrast to not modify, or to only minimally modify, most parameters.

### Compositions

The estetrol component of the present invention encompasses substances selected from the group consisting of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms; and combinations thereof. More preferably, the estetrol component is estetrol (including estetrol hydrates). Most preferably, the estetrol component contained in the dosage unit is estetrol monohydrate.

The estetrol component of the invention is used at a daily dose equivalent to from about 15 mg to about 25 mg of estetrol monohydrate. In other words, when the estetrol component is not estetrol monohydrate itself, the daily dose of the estetrol component is adjusted to yield a therapeutic effect equivalent to that of a daily dose of about 15 mg to about 25 mg of estetrol monohydrate.

In a particularly preferred embodiment of the invention the pharmaceutical composition according to invention is designed for daily administration, i.e. it represents a daily dosage unit. In the case of oral administration, the oral dosage unit according to the invention is preferably a solid or semi-solid dosage form such as tablets, capsules, cachets, pellets, pills, powders and granules. The term “solid or semi-solid dosage form” also encompasses capsules that contain a liquid, e.g. an oil, in which the present estetrol component and/or the optional progestogenic component is dissolved or dispersed. Tablets and equivalent solid and semi-solid dosage forms can suitably contain materials such as binders (e.g. hydroxypropylmethyl cellulose, polyvinyl pyrrolidone, other cellulosic materials and starch), diluents (e.g. lactose and other sugars, starch, dicalcium phosphate and cellulosic materials), disintegrating agents (e.g. starch polymers and cellulosic materials) and lubricating agents (e.g., stearates and talc). These tablets and equivalent solid dosage forms may be prepared by wet granulation, e.g. using an aqueous solution or an organic solution, as well as by direct compression.

In the case of sublingual, buccal or sublabial administration, the pharmaceutical composition according to the invention is preferably an orodispersible dosage unit.

The term “orodispersible dosage unit” as used herein refers to a dosage unit that is designed to rapidly disintegrate in the oral cavity when it comes into contact with saliva and to disperse the estetrol component into the saliva so it may be absorbed through the mucosal lining of the oral cavity.

When the dosage unit is an orodispersible dosage unit, the rate of release of the estetrol component from the dosage unit can suitably be determined using the disintegration test according to Ph. Eur. 2.9.1 (“Disintegration of tablets and capsules”) and USP <701> (“Disintegration”), for example using water as the disintegration medium. An orodispersible solid dosage unit of the present invention, when subjected to the aforementioned disintegration test, typically disintegrates within less than 5 minutes, more preferably within less than 2 minutes, still more preferably within less than 1,5 minutes, still more preferably within less than 1 minute, still more preferably within less than 45 seconds, and most preferably within less than 30 seconds.

When the patient still has a uterus, an optional progestogenic component may be administered in addition to the estetrol component.

Examples of progestogenic components which may suitably be used in accordance with the present invention include: levonorgestrel, norgestimate, norethisterone, dydrogesterone, drospirenone, 3-beta-hydroxydesogestrel, 3-ketodesogestrel, 17-deacetylnorgestimate, 19-norprogesterone, acetoxypregnenolone, allylestrenol, amgestone, chlormadinone, cyproterone, demegestone, desogestrel, dienogest, dihydrogesterone, dimethisterone, ethisterone, ethynodiol diacetate, fluorogestone acetate, gastrinone, gestodene, gestrinone, hydroxymethylprogesterone, hydroxyprogesterone, lynestrenol, mecirogestone, medroxyprogesterone, megestrol, mele,gestrol, nomegestrol, norethindrone, norethynodrel, norgestrel (including d-norgestrel, and dl-norgestrel), norgestrienone, normethisterone, progesterone, quingestanol, (17 alpha)-17-hydroxy-11-methylene-19-norpregna-4, 15-dien-20-yn-3-one, tibolone, trimegestone, algestone-acetophenide, nestorone, promegestone, 17-hydroxyprogesterone esters, 19-nor-17hydroxyprogesterone, 17alpha-ethynyltestosterone, 17alpha-ethynil-19-nortestosterone, d-17beta-acetoxy-13beta-ethyl-17alpha-ethynylgon-4-en-3-one oxime, 6beta, 7beta; 15beta, 16beta-dimethylene-3-oxo-17-pregna-4,9 (11)-diene-21, 17beta-carbolactone or tanaproget and precursors of these compounds that are capable of liberating these progestogens in vivo when used in the present method.

Preferably the progestogenic component used in the present method is selected from the group consisting of progesterone, drospirenone, dydrogesterone, precursors of these progestogens and mixtures thereof.

In one embodiment, the invention provides a combination composition comprising an estetrol component together with progesterone.

In one embodiment, the invention provides a combination composition comprising an estetrol component together with drospirenone.

In one embodiment, the invention provides a combination composition comprising an estetrol component together with dydrogesterone.

When the progestogenic component of the invention is drospirenone, it is preferably used at a daily dose of from 0.5 mg to 10 mg, even more preferably of from 1 mg to 4 mg.

When the progestogenic component of the invention is dydrogesterone, it is preferably used at a daily dose of about 5 mg to about 10 mg, more preferably at a daily dose of about 5 mg.

When the progestogenic component of the invention is progesterone, it is preferably used at a daily dose of from 50 mg to 200 mg. In one embodiment, progesterone is used at a daily dose of 50 mg to 100 mg when it is used continuously. In another embodiment, progesterone is used at a daily dose of 100 mg to 200 mg when it is used sequentially, for example when it is administered during about 14 days every month.

When a different progestogenic component is used, the daily dose is adjusted such as to give the same pharmacological effect as a dose of 50 mg to 200 mg of progesterone.

In a preferred embodiment of the invention, the composition combines the estetrol component and the optional progestogenic component into a single dosage unit, preferably a daily dosage unit. In a more preferred embodiment of the invention, said combined daily dosage unit is an oral combined daily dosage unit.

In one embodiment, the invention provides an oral combined daily dosage unit comprising an estetrol component and progesterone.

In one embodiment, the invention provides an oral combined daily dosage unit comprising an estetrol component and drospirenone.

In one embodiment, the invention provides an oral combined daily dosage unit comprising an estetrol component and dydrogesterone.

In a preferred embodiment of the invention, an oral combined daily dosage unit combining estetrol at a daily dose of about 20 mg with progesterone at a daily dose of about 100 mg is provided.

In another embodiment of the invention, the estetrol component is administered to a patient who still has a uterus in conjunction with a Selective Estrogen Receptor Modulator (SERM), in particular in conjunction with bazedoxifene. Preferably bazedoxifene is administered at a daily dose of about 10 mg to 50 mg. More preferably, bazedoxifene is administered at a daily dose of about 20 mg.

In one embodiment, the invention provides a combination therapy comprising an estetrol component together with bazedoxifene.

In one embodiment, the invention provides an oral combined daily dosage unit comprising an estetrol component and bazedoxifene.

The present invention has been described above with reference to a number of exemplary embodiments. Modifications and alternative implementations of some parts or elements are possible, and are included in the scope of protection as defined in the appended claims.

### EXAMPLES

### Example 1—A Dose-Finding Study to Select the Daily Oral Dose of Estetrol (E4) for the Treatment of Vasomotor Symptoms in Post-Menopausal Women

### Study Enrolment and Duration

Enrolment was approximately 18 months. Individual subject participation was up to 27 weeks: up to 6 weeks pre-screening and washout, up to 4 weeks screening and run-in period, up to 91 days (13 weeks) of E4/placebo treatment followed by 2 weeks (14 days) of progestin therapy and a Follow up visit 1 week after completion of progestin therapy in non-hysterectomied subjects only.

### Primary Efficacy Objective

To define the minimum effective dose (MED) of the oral dose of E4 by evaluating changes in frequency and in severity of moderate to severe vasomotor symptoms (VMS).

### Methodology

This was a prospective, multicentre, randomised, placebo-controlled, double-blinded, dose-finding study.

### Subject Population

Eligible subjects were hysterectomied and non-hysterectomied post-menopausal women aged 40 to 65 years, inclusive, presenting at least 7 moderate to severe hot flushes/day or at least 50 moderate to severe hot flushes/week.

### Diagnosis and Inclusion Criteria

The subjects have met all of the following inclusion criteria at the randomization visit. These criteria were assessed during the screening period:

1. Women aged 40 to 65 years, inclusive, presenting at least 7 moderate to severe hot flushes/day or at least 50 moderate to severe hot flushes/week in the week preceding randomization.

2. Body Mass Index (BMI) between 18.0 and 35.0 kg/m², inclusive.

3. Post-menopausal status defined as levels of follicle stimulating hormone (FSH) >40 IU/L and:
amenorrhoea for at least 12 consecutive months or,
amenorrhoea for at least 6 months with estradiol (E2) <20 pg/mL or,
at least 6 weeks post-surgical bilateral oophorectomy with or without hysterectomy with a copy of the pathology report or a statement on letterhead from the subject's physician documenting both ovaries were removed is required.

4. For non-hysterectomiaed women: intact uterus with bi-layer endometrial thickness ≤5 mm on TVUS.

5. Negative pregnancy test.

6. Good physical and mental health, in the judgement of the Principal Investigator (PI), on the basis of medical, surgical and gynaecological history, physical examination, gynaecological examination, clinical laboratory, and vital signs.

7. Subject has provided signed and dated written informed consent before admission to the study.

8. Subject is able to understand and comply with the protocol requirements, instructions, and protocol-stated restrictions.

### Exclusion Criteria

Potential study subjects were excluded if one of the following exclusion criteria was present at the randomization visit. These criteria were assessed during the screening period:

1. For non-hysterectomied women: uterine disease or medical condition including:
a. Bi-layer endometrial thickness >5 mm as determined by TVUS;
b. Presence of fibroid(s) that obscure(s) evaluation of endometrium by TVUS;
c. History or presence of uterine cancer;
d. Presence of endometrial hyperplasia;
e. Presence of an endometrial polyp with hyperplastic or malignant epithelium.

2. Undiagnosed vaginal bleeding in the last 12 months.

3. Any history of malignancy with the exception of basal cell (excluded if within the prior 2 years) or squamous cell (excluded if within the prior one year) carcinoma of the skin. Any clinically significant findings at the breast examination and/or on mammography suspicious of breast malignancy that would require additional clinical testing to rule out breast cancer (however, simple cysts confirmed by ultrasound were allowed). Note: A screening mammogram was required unless the subject had a written documentation of a mammogram performed within the last 9 months.

4. Abnormal cervical Pap smear in non-hysterectomied subjects (written documentation of prior test within 18 months or test at screening exam) with evidence of cervical dysplasia greater than low grade squamous intraepithelial lesion (LSIL). Women with a diagnosis of atypical squamous cells of undetermined significance (ASCUS) were enrolled.

5. Systolic blood pressure (BP) outside the range 90 to 140 mmHg, diastolic BP outside the range 60 to 90 mmHg, and/or heart rate outside the range 40 to 100 bpm. Subjects with mild to moderate hypertension who were controlled on a stable antihypertension regimen were enrolled if they met the inclusion/exclusion criteria.

6. Any clinically significant abnormality identified on the screening 12-lead ECG.

7. History of venous or arterial thromboembolic disease (e.g., deep vein thrombosis, pulmonary embolism, stroke, myocardial infarction, angina pectoris, etc.), history of known coagulopathy or abnormal coagulation factors.

8. Diabetes mellitus with poor glycaemic control in the last 6 months assessed by laboratory values of glucose outside the normal ranges and glycated haemoglobin above 7%.

9. Dyslipoproteinaemia predisposes the subject to atherosclerotic cardiovascular disease (ASCVD). If a subject had a 10 years ASCVD score ≥5% as calculated using the ASCVD risk estimator (ACC/AHA Cardiovascular risk assessment guideline, 2013), she was not be included in the trial. In all cases, LDL cholesterol level ≥190 mg/dL or triglycerides plasma level >400 mg/dL were exclusionary.

If a subject was receiving a lipid-lowering therapy, her treatment had to be on a stable dose for at least 1 month before screening and the same eligibility criteria had to be used.

10. Smoking >10 cigarettes/day or use of >1 ml/day of nicotine containing liquid for electronic cigarette.

11. Presence or history of gallbladder disease, unless cholecystectomy had been performed.

12. Systemic lupus erythematosus.

13. Multiple sclerosis.

14. Acute or chronic liver disease.

15. Acute or chronic renal impairment, including severe renal impairment.

16. Uncontrolled thyroid disorders.

17. Subject had a history of major depression or post-traumatic stress disorder (PTSD) within 2 years, OR a history of other major psychiatric disorder at any time (e.g., schizophrenia, bipolar disorder, etc.).

18. Use of oestrogen or progestin containing drug(s). A washout period is required before the Run-in Period in case of use of:
a. Vaginal hormonal products (rings, creams, gels): washout of at least 4 weeks;
b. Transdermal oestrogen or oestrogen/progestin: washout of at least 4 weeks;
c. Oral oestrogen and/or progestin: washout of at least 4 weeks;
d. Intrauterine progestin therapy: washout of at least 4 weeks;

Current users of progestin implants or oestrogen alone injectable drug therapy were not allowed to participate unless the treatment was stopped more than 3 months ago. Current users of oestrogen pellet therapy or progestin injectable drug therapy were not allowed to participate unless the treatment was stopped more than 6 months ago.

19. Use of non-hormonal treatments to reduce hot flushes. A washout period of 1 week was required before the Run-in Period in the case of use of non-hormonal prescription and over-the-counter (OTC) treatments for hot flushes (such as anti-depressants paroxetine, escitalopram, venlafaxine, desvenlafaxine, and clonidine; or phytoestrogens, black cohosh, etc.). If one of these treatments was concomitantly taken with an oestrogen or progestin-containing drug, washout periods could be combined and did not have to be sequential.

20. Use of medication that may affect the outcome of the VMS endpoints within 28 days before the Run-in Period. This included (but was not limited to): SSRIs [selective serotonin reuptake inhibitors], SNRIs [serotonin and norepinephrine reuptake inhibitors], dopaminergic or antidopaminergic drugs, or gabapentin.

21. History or presence of allergy to the investigational product or drugs of this class, or history of drug or other allergy that, in the opinion of the Investigator contraindicated subject participation.

22. History or presence of allergy or intolerance to any component of the investigational product.

23. History of alcohol or substance abuse or dependence in the 12 months as determined by the Investigator, i.e. subject consumed excessive alcohol, abused drugs, or had a condition that could compromise the subject's ability to comply with study requirements in the Investigator's opinion.

24. Sponsor or Contract Research Organization (CRO) employees, or personnel in the department of the Investigator and relatives affiliated with this study.

25. Subjects with porphyria and subjects with known or suspected history of a clinically significant systemic disease, unstable medical disorders, life-threatening disease or current malignancies that would pose a risk to the subject in the opinion of the Investigator.

26. Participation in another investigational drug clinical study within 1 month (30 days) or had received an investigational drug within the last 3 months (90 days).

27. Was judged by the Investigator to be unsuitable for any reason.

### Number of Subjects

### Intention-to-Treat Principle

This principle asserts that the effect of a treatment policy can be best assessed by evaluating on the basis of the intention to treat a subject (i.e. the planned treatment regimen) rather than the actual treatment given. It has the consequence that subjects allocated to a treatment group should be followed up, assessed and analysed as members of that group irrespective of their compliance to the planned course of treatment.

Further, the intention-to-treat principle implies that the primary analysis should include all randomised subjects.

Preservation of the initial randomisation in analysis is important in preventing bias and in providing a secure foundation for statistical tests. In many clinical trials the use of the full analysis set provides a conservative strategy. Under many circumstances it may also provide estimates of treatment effects which are more likely to mirror those observed in subsequent practice.

In the present study, the Intention-To-Treat group included a total of 257 patients.

Subjects were randomly allocated to one of the 5 treatment arms in a 1:1:1:1:1 ratio. The randomisation was stratified by centre.

### Study Visits

| | | Notation | |
|---|---|---|---|
| | | as used | |
| | | throughout | |
| | | all tables, | |
| | | listings | |
| Week | Visit | and FIGS. | Study part |
| Week −7 to −4 | Visit 1 | V1 | Screening |
| | (Pre-Screening) | | |
| Week −4 to −3 | Visit 1a | V1a | Screening |
| | (Screening) | | |
| Week −1 | Visit 2 | V2 | Randomisation |
| | (Baseline) | | |
| Week 5 | Visit 3 | V3 | Treatment period |
| Week 13 | Visit 4 (EOT) | V4 | End of treatment |
| Week 16 | Visit 5 (EOS) | V5 | End of study |

### Test Product and Reference Therapy, Dose, and Mode of Administration

All treatments (Estetrol, hereinafter E4, [2.5 mg, 5 mg, 10 mg, 15 mg] capsule) were administered once daily (QD) per os for at least 12 consecutive weeks until the last biological assessments (Day 91 maximum) had been performed.

Placebo, 1 capsule administered QD per os for at least 12 consecutive weeks until the last biological assessments (Day 90 maximum) have been performed.

If during the course of the trial, a double layer endometrial thickness ≥15 mm was detected on TVUS and/or abnormal uterine bleeding (in the judgement of the gynaecologist in light of the oestrogen therapy) was reported by a non-hysterectomied woman, she underwent an endometrial biopsy and was treated with progestin (10 mg dydrogesterone) QD until end of Week 11 in a sequential way (i.e., a 14 day progestin treatment period followed by a 14 day progestin treatment pause) in addition to the E4/placebo treatment. If the endometrial biopsy showed endometrial hyperplasia, the subject participation was immediately stopped and the treatment of hyperplasia was performed as per local guidelines. If an abnormal uterine bleeding occured again after a first normal endometrial biopsy, a thorough gynaecological examination and a TVUS were performed. If necessary in the judgment of the gynaecologist, a second endometrial biopsy was performed. After the E4/Placebo treatment period, all non-hysterectomied subjects (including those having received the progestin previously) received progestin therapy for 14 days with 10 mg dydrogesterone QD.

### Results

### A. Vasomotors Parameters for Each of the 5 Treatment Groups

a. VMS Frequency
i. Absolute Change (Mean Change from Baseline) in Weekly Frequency of Moderate to Severe VMS
a) Week-by-week for each group

| | 2.5 mg | | 5 mg | | 10 mg | | 15 mg | | Placebo | |
|---|---|---|---|---|---|---|---|---|---|---|
| Week | Mean | SD | Mean | SD | Mean | SD | Mean | SD | Mean | SD |
| 0 | 0.00 | 0.00 | 0.00 | 0.00 | 0.00 | 0.00 | 0.00 | 0.00 | 0.00 | 0.00 |
| 1 | −17.28 | 19.71 | −15.04 | 17.36 | −13.37 | 19.59 | −14.86 | 16.45 | −16.65 | 16.24 |
| 2 | −25.10 | 23.91 | −21.68 | 19.26 | −23.47 | 24.74 | −29.19 | 20.25 | −27.38 | 23.16 |
| 3 | −32.46 | 26.15 | −24.38 | 21.36 | −31.34 | 25.21 | −36.30 | 23.23 | −30.16 | 23.09 |
| 4 | −35.89 | 31.57 | −27.57 | 22.47 | −36.38 | 22.62 | −41.43 | 21.60 | −32.94 | 23.14 |
| 5 | −37.16 | 33.27 | −30.70 | 23.05 | −39.07 | 21.55 | −44.34 | 20.95 | −34.70 | 23.01 |
| 6 | −39.98 | 35.44 | −38.02 | 22.13 | −42.56 | 21.18 | −46.58 | 19.83 | −37.18 | 21.25 |
| 7 | −42.44 | 36.96 | −38.23 | 21.73 | −43.30 | 22.18 | −48.51 | 19.34 | −38.55 | 21.85 |
| 8 | −43.62 | 38.43 | −38.55 | 23.54 | −44.46 | 23.30 | −48.35 | 19.75 | −38.13 | 21.69 |
| 9 | −45.19 | 36.91 | −39.34 | 23.51 | −45.19 | 24.22 | −49.62 | 18.97 | −39.25 | 22.74 |
| 10 | −45.54 | 37.93 | −39.83 | 23.79 | −45.93 | 23.58 | −48.88 | 20.04 | −41.12 | 21.82 |
| 11 | −44.74 | 39.30 | −41.46 | 23.59 | −45.90 | 23.57 | −49.92 | 19.42 | −42.50 | 22.68 |
| 12 | −45.04 | 38.91 | −40.60 | 24.37 | −47.21 | 22.87 | −50.94 | 18.38 | −42.97 | 22.31 |

In order to analyse the data recorded in this study, treatment groups have been compared using an ANCOVA (analysis of covariance) with respect to the change in weekly frequency of moderate to severe VMS from baseline to weeks 4 and 12. The ANCOVA model includes treatment (“trt1”) and study centre (“SITEPOOL”) as a fixed effect and baseline (“base”) as a covariate.
b) Covariate significance

The Table below presents a comparison over all treatment groups.

| Analysis | | |
|---|---|---|
| Timepoint | | |
| (N) | Covariate | p_value |
| 4 | trt1 | 0.0164 |
| 4 | base | <.0001 |
| 4 | SITEPOOL | 0.2214 |
| 12 | trt1 | 0.0384 |
| 12 | base | <.0001 |
| 12 | SITEPOOL | 0.2706 |

Upon review, it was considered that the effect due to the study centre was not very important, and a second ANCOVA was performed without accounting for study centres.

### Without Site Effect

| Analysis | | |
|---|---|---|
| Timepoint | | |
| (N) | Covariate | p_value |
| 4 | trt1 | 0.0130 |
| 4 | base | <.0001 |
| 12 | trt1 | 0.0254 |
| 12 | base | <.0001 |

c) The Table below presents mean change from baseline by week and treatment:

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −33.7959277 | −40.006133 | −27.585722 |
| 4 | 2.5 | −32.2601479 | −38.742792 | −25.777504 |
| 4 | 5 | −27.4372730 | −34.135693 | −20.738853 |
| 4 | 10 | −35.3672857 | −41.716169 | −29.018402 |
| 4 | 15 | −43.5996056 | −50.256441 | −36.942770 |
| 12 | 0 | −43.7344395 | −50.150527 | −37.318352 |
| 12 | 2.5 | −40.1310220 | −46.828579 | −33.433465 |
| 12 | 5 | −40.6802008 | −47.600688 | −33.759714 |
| 12 | 10 | −45.8376026 | −52.396965 | −39.278240 |
| 12 | 15 | −53.8273074 | −60.704831 | −46.949784 |

### Without Site Effect

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −33.6519764 | −39.821576 | −27.482377 |
| 4 | 2.5 | −32.6798943 | −39.056255 | −26.303533 |
| 4 | 5 | −27.8705668 | −34.540176 | −21.200958 |
| 4 | 10 | −35.8741722 | −42.156516 | −29.591828 |
| 4 | 15 | −44.3569892 | −50.965323 | −37.748656 |
| 12 | 0 | −43.9304437 | −50.292513 | −37.568374 |
| 12 | 2.5 | −40.6840423 | −47.259323 | −34.108761 |
| 12 | 5 | −41.0122585 | −47.889936 | −34.134581 |
| 12 | 10 | −46.5333871 | −53.011718 | −40.055056 |
| 12 | 15 | −54.9073270 | −61.721817 | −48.092837 |

All statistical tests are supported by presenting Least Square adjusted mean (LS adjusted mean: the group means after having controlled for a covariate; also referred to as marginal means or estimated marginal means) and 95% confidence intervals for the respective treatment effects.

These LS adjusted means and Confidence Intervals are based on the statistical models used for the analysis.

The Confidence Intervals implies that if the same population is sampled on numerous occasions and interval estimates are made on each occasion, the resulting intervals would bracket the true population parameter in approximately 95% of the cases.
d) The Table below presents differences with placebo by week and treatment

| Analysis | | | LS | Lower 95% | Upper 95% | |
|---|---|---|---|---|---|---|
| Timepoint | Higher | Lower | adjusted | confidence | confidence | |
| (N) | dose (mg) | dose (mg) | mean | limit | limit | p_value |
| 4 | 2.5 | 0 | 1.535780 | −9.602669 | 12.674228 | 0.99183 |
| 4 | 5.0 | 0 | 6.358655 | −4.958071 | 17.675380 | 0.44910 |
| 4 | 10.0 | 0 | −1.571358 | −12.573355 | 9.430639 | 0.99067 |
| 4 | 15.0 | 0 | −9.803678 | −21.033229 | 1.425873 | 0.10653 |
| 12 | 2.5 | 0 | 3.603418 | −7.904295 | 15.111130 | 0.86103 |
| 12 | 5.0 | 0 | 3.054239 | −8.637660 | 14.746138 | 0.92091 |
| 12 | 10.0 | 0 | −2.103163 | −13.469900 | 9.263574 | 0.97581 |
| 12 | 15.0 | 0 | −10.092868 | −21.694703 | 1.508967 | 0.10838 |

### Without Site Effect

| Analysis | | | | Lower 95% | Upper 95% | |
|---|---|---|---|---|---|---|
| Timepoint | Higher | Lower | LS adjusted | confidence | confidence | |
| (N) | dose (mg) | dose (mg) | mean | limit | limit | p_value |
| 4 | 2.5 | 0 | 0.972082 | −10.164048 | 12.108212 | 0.99860 |
| 4 | 5.0 | 0 | 5.781410 | −5.579628 | 17.142447 | 0.53888 |
| 4 | 10.0 | 0 | −2.222196 | −13.242056 | 8.797664 | 0.96728 |
| 4 | 15.0 | 0 | −10.705013 | −21.979289 | 0.569264 | 0.06834 |
| 12 | 2.5 | 0 | 3.246401 | −8.237137 | 14.729940 | 0.89864 |
| 12 | 5.0 | 0 | 2.918185 | −8.797276 | 14.633647 | 0.93259 |
| 12 | 10.0 | 0 | −2.602943 | −13.966584 | 8.760697 | 0.94911 |
| 12 | 15.0 | 0 | −10.976883 | −22.602878 | 0.649111 | 0.07057 |

From these Tables, it can be seen that the 15 mg daily dose generates a near statistically significant difference with placebo at 4 weeks (p-value of 0.10653 in the first statistical analysis, and of 0.06834 in the analysis without pooled site) and at 12 weeks (p-value of 0.10838 in the first statistical analysis, and of 0.07057 in the analysis without pooled site).

ii. Relative change (% from baseline) in weekly frequency of moderate to severe VMS
a) Week-by-week for each group

| | 2.5 mg | | 5 mg | | 10 mg | | 15 mg | | Placebo | |
|---|---|---|---|---|---|---|---|---|---|---|
| Week | Mean | SD | Mean | SD | Mean | SD | Mean | SD | Mean | SD |
| 0 | 0% | 0% | 0% | 0% | 0% | 0% | 0% | 0% | 0% | 0% |
| 1 | −23% | 23% | −23% | 28% | −21% | 29% | −22% | 33% | −26% | 25% |
| 2 | −34% | 27% | −33% | 29% | −37% | 37% | −46% | 34% | −41% | 35% |
| 3 | −44% | 29% | −38% | 33% | −48% | 39% | −57% | 34% | −45% | 33% |
| 4 | −49% | 33% | −43% | 34% | −55% | 35% | −67% | 30% | −49% | 32% |
| 5 | −50% | 33% | −47% | 34% | −60% | 33% | −72% | 28% | −52% | 32% |
| 6 | −54% | 36% | −57% | 31% | −65% | 33% | −76% | 26% | −56% | 30% |
| 7 | −57% | 36% | −57% | 30% | −66% | 34% | −79% | 25% | −58% | 30% |
| 8 | −58% | 38% | −57% | 32% | −68% | 35% | −79% | 26% | −58% | 31% |
| 9 | −61% | 36% | −59% | 32% | −69% | 36% | −81% | 24% | −60% | 31% |
| 10 | −61% | 37% | −60% | 32% | −70% | 35% | −80% | 27% | −63% | 30% |
| 11 | −60% | 39% | −63% | 31% | −70% | 34% | −82% | 25% | −65% | 31% |
| 12 | −61% | 38% | −62% | 32% | −72% | 33% | −84% | 23% | −65% | 30% |

From this table, it can be seen that the 15 mg daily dose resulted in a reduction of over 80% in the frequency of moderate to severe VMS when compared to baseline.
b) Covariate significance

The Table below presents a comparison over all treatment groups.

| Analysis | | |
|---|---|---|
| Timepoint | | |
| (N) | Covariate | p_value |
| 4 | trt1 | 0.0147 |
| 4 | base | 0.3684 |
| 4 | SITEPOOL | 0.3236 |
| 12 | trt1 | 0.0100 |
| 12 | base | 0.1490 |
| 12 | SITEPOOL | 0.0958 |

### Without Site Enlect

| Analysis | | |
|---|---|---|
| Timepoint | | |
| (N) | Covariate | p_value |
| 4 | trt1 | 0.0107 |
| 4 | base | 0.2264 |
| 12 | trt1 | 0.0065 |
| 12 | base | 0.0774 |

c) The Table below presents mean relative change (%) from baseline by wook and treatment

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.49080693 | −0.578953 | −0.402661 |
| 4 | 2.5 | −0.48482309 | −0.576836 | −0.392811 |
| 4 | 5 | −0.42247695 | −0.517552 | −0.327402 |
| 4 | 10 | −0.54456428 | −0.634678 | −0.454450 |
| 4 | 15 | −0.64721795 | −0.741703 | −0.552733 |
| 12 | 0 | −0.64237949 | −0.726371 | −0.558388 |
| 12 | 2.5 | −0.60565210 | −0.693328 | −0.517976 |
| 12 | 5 | −0.60259514 | −0.693189 | −0.512001 |
| 12 | 10 | −0.70041629 | −0.786283 | −0.614549 |
| 12 | 15 | −0.80089486 | −0.890927 | −0.710863 |

### Without Site Effect

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.48986685 | −0.577115 | −0.402619 |
| 4 | 2.5 | −0.49532213 | −0.585494 | −0.405151 |
| 4 | 5 | −0.43132298 | −0.525642 | −0.337004 |
| 4 | 10 | −0.55503454 | −0.643877 | −0.466192 |
| 4 | 15 | −0.66014529 | −0.753597 | −0.566693 |
| 12 | 0 | −0.65107969 | −0.735113 | −0.567046 |
| 12 | 2.5 | −0.62210640 | −0.708956 | −0.535256 |
| 12 | 5 | −0.61436957 | −0.705214 | −0.523525 |
| 12 | 10 | −0.71732171 | −0.802891 | −0.631752 |
| 12 | 15 | −0.82338275 | −0.913392 | −0.733373 |

d) The Table below presents differences with placebo by week and treatment

| Anal- | | | | | | |
|---|---|---|---|---|---|---|
| ysis | | | | Lower | Upper | |
| Time- | Higher | Lower | LS | 95% | 95% | |
| point | dose | dose | adjusted | confidence | confidence | |
| (N) | (mg) | (mg) | mean | limit | limit | p_value |
| 4 | 2.5 | 0 | 0.005984 | −0.152112 | 0.164079 | 0.99995 |
| 4 | 5.0 | 0 | 0.068330 | −0.092296 | 0.228956 | 0.68478 |
| 4 | 10.0 | 0 | −0.053757 | −0.209916 | 0.102401 | 0.81738 |
| 4 | 15.0 | 0 | −0.156411 | −0.315800 | 0.002977 | 0.05622 |
| 12 | 2.5 | 0 | 0.036727 | −0.113917 | 0.187372 | 0.93696 |
| 12 | 5.0 | 0 | 0.039784 | −0.113271 | 0.192840 | 0.92218 |
| 12 | 10.0 | 0 | −0.058037 | −0.206836 | 0.090762 | 0.74506 |
| 12 | 15.0 | 0 | −0.158515 | −0.310392 | −0.006639 | 0.03771 |

From this Table it can be seen that the 15 mg daily dose generates a statistically significant difference with placebo at 12 weeks (p-value of 0.03771) and an almost statistically significant difference with placebo at 4 weeks (p-value of 0.05622).

It is particularly striking to observe the low p-values obtained for the 15 mg dose by comparison to the elevated p-values obtained for the 10 mg dose.

### Without Site Effect

| Anal- | | | | | | |
|---|---|---|---|---|---|---|
| ysis | | | | Lower | Upper | |
| Time- | Higher | Lower | LS | 95% | 95% | |
| point | dose | dose | adjusted | confidence | confidence | |
| (N) | (mg) | (mg) | mean | limit | limit | p_value |
| 4 | 2.5 | 0 | −0.005455 | −0.162937 | 0.152027 | 0.99996 |
| 4 | 5.0 | 0 | 0.058544 | −0.102119 | 0.219206 | 0.78717 |
| 4 | 10.0 | 0 | −0.065168 | −0.221006 | 0.090670 | 0.69797 |
| 4 | 15.0 | 0 | −0.170278 | −0.329714 | −0.010843 | 0.03206 |
| 12 | 2.5 | 0 | 0.028973 | −0.122708 | 0.180654 | 0.97301 |
| 12 | 5.0 | 0 | 0.036710 | −0.118034 | 0.191454 | 0.94271 |
| 12 | 10.0 | 0 | −0.066242 | −0.216339 | 0.083855 | 0.65761 |
| 12 | 15.0 | 0 | −0.172303 | −0.325866 | −0.018741 | 0.02210 |

From this Table it can be seen that in the statistical analysis not accounting for site effects, the 15 mg daily dose generates a statistically significant difference with placebo at 4 weeks (p=0.03206) and at 12 weeks (p=0.02210).

It is particularly striking to observe the low p-values obtained for the 15 mg dose by comparison to the elevated p-values obtained for the 10 mg dose.

iii. Frequency Changes in Groups of Responders in Weekly Frequency of Moderate to Severe VMS

VMS frequency was also studied by grouping patients according to their degree of response. A first grouping of patients showing a response of 50% or more (relative change from baseline) was prepared. According to this analysis, at week 12, the 15 mg daily dose group contains 91.8% of responders, while the placebo group contains 65.5% of responders. The difference between these two groups has a p-value below 0.01, whereas the difference between the 10 mg daily dose group and the placebo group is not statistically significant (p value>0.1).

A second grouping of patients showing a response of 75% or more was prepared. According to this analysis, at week 12, the 15 mg daily dose group contains 77.6% of responders, while the placebo group contains 43.6% of responders. The difference between these two group has a p-value below 0.001, whereas the difference between the 10 mg daily dose group and the placebo group is not statistically significant (p value>0.05).

b. VMS Severity
i. Absolute Change (Mean Change from Baseline) in Weekly Severity of Moderate to Severe VMS
a) Week-by-week for each group

| | 2.5 mg | 5 mg | 10 mg | 15 mg | Placebo |
|---|---|---|---|---|---|
| Week | Mean | Mean | Mean | Mean | Mean |
| 0 | 0 | 0 | 0 | 0 | 0 |
| 1 | −0.1542 | −0.098 | −0.2028 | −0.072 | −0.1638 |
| 2 | −0.2112 | −0.1199 | −0.3007 | −0.2853 | −0.2746 |
| 3 | −0.3246 | −0.1574 | −0.3983 | −0.4842 | −0.2676 |
| 4 | −0.3373 | −0.2341 | −0.4842 | −0.5897 | −0.3327 |
| 5 | −0.3984 | −0.1683 | −0.5107 | −0.7432 | −0.4146 |
| 6 | −0.5014 | −0.3212 | −0.6086 | −0.7035 | −0.45 |
| 7 | −0.4562 | −0.2787 | −0.643 | −0.8112 | −0.4645 |
| 8 | −0.5359 | −0.3239 | −0.6644 | −0.8854 | −0.52 |
| 9 | −0.5465 | −0.3249 | −0.6864 | −0.9092 | −0.5522 |
| 10 | −0.6052 | −0.3667 | −0.709 | −0.9628 | −0.5514 |
| 11 | −0.5564 | −0.4161 | −0.666 | −1.0123 | −0.6551 |
| 12 | −0.6279 | −0.4007 | −0.6941 | −1.0425 | −0.6604 |

In order to better analyse the data recorded in this study, treatment groups have been compared using an ANCOVA (analysis of covariance) with respect to the change in severity of moderate to severe VMS from baseline to mild, moderate and severe VMS at week 4 and week 12 for each active treatment versus placebo. For women who experienced 100% VMS relief at week 4 and/or week 12, a value of zero was attributed. The ANCOVA model includes treatment (“trt1”) as a fixed effect and baseline (“base”) as a covariate.
b) Covariate significance

The Table below presents a comparison over all treatment groups.

| Analysis | | |
|---|---|---|
| Timepoint | | |
| (N) | Covariate | p_value |
| 4 | trt1 | 0.0119 |
| 4 | base | 0.3781 |
| 12 | trt1 | 0.0032 |
| 12 | base | 0.7990 |

c) The Table below presents mean change from baseline by week and treatment:

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.327801 | −0.470747 | −0.184854 |
| 4 | 2.5 | −0.341092 | −0.486518 | −0.195666 |
| 4 | 5 | −0.238552 | −0.393041 | −0.084063 |
| 4 | 10 | −0.482536 | −0.627769 | −0.337303 |
| 4 | 15 | −0.588755 | −0.739764 | −0.437746 |
| 12 | 0 | −0.658280 | −0.868694 | −0.447865 |
| 12 | 2.5 | −0.629592 | −0.843656 | −0.415528 |
| 12 | 5 | −0.402567 | −0.629971 | −0.175163 |
| 12 | 10 | −0.693404 | −0.907183 | −0.479624 |
| 12 | 15 | −1.042087 | −1.264369 | −0.819805 |

d) The Table below presents differences with placebo by week and treatment

| Anal- | | | | | | |
|---|---|---|---|---|---|---|
| ysis | | | | Lower | Upper | |
| Time- | Higher | Lower | LS | 95% | 95% | |
| point | dose | dose | adjusted | confidence | confidence | |
| (N) | (mg) | (mg) | mean | limit | limit | p_value |
| 4 | 2.5 | 0 | −0.013291 | −0.268767 | 0.242185 | 0.9998 |
| 4 | 5.0 | 0 | 0.089249 | −0.174507 | 0.353004 | 0.8253 |
| 4 | 10.0 | 0 | −0.154735 | −0.409222 | 0.099751 | 0.3767 |
| 4 | 15.0 | 0 | −0.260954 | −0.520749 | −0.001159 | 0.0486 |
| 12 | 2.5 | 0 | 0.028687 | −0.347368 | 0.404743 | 0.9992 |
| 12 | 5.0 | 0 | 0.255713 | −0.132530 | 0.643955 | 0.3062 |
| 12 | 10.0 | 0 | −0.035124 | −0.409723 | 0.339475 | 0.9981 |
| 12 | 15.0 | 0 | −0.383807 | −0.766221 | −0.001394 | 0.0489 |

From this Table it can be seen that the 15 mg daily dose generates a statistically significant difference with placebo at 4 weeks (p-value of 0.0486) and at 12 weeks (p-value of 0.0489). The 15 mg dose thus significantly improves the severity of VMS at weeks 4 and 12 compared with placebo.

For the severity parameter also, the difference between the 10 mg and 15 mg doses is impressive: this is reflected firstly in the mean changes from baseline presented in the Table of section c) above, where for example at 12 weeks the LS adjusted mean for 10 mg is −0.69 (to be compared with −0.66 found for the placebo group), while it is −1.04 for the 15 mg daily dose group. This clear difference is mirrored by a near 8-fold improvement in the p-value at 4 weeks when switching from 10 mg to 15 mg per day, and an over 20-fold improvement in the p-value at 12 weeks when switching from 10 mg to 15 mg per day.

ii. Relative Change (% from Baseline) in Weekly Severity of Moderate to Severe VMS
a) Week-by-week for each group

| | 2.5 mg | 5 mg | 10 mg | 15 mg | Placebo |
|---|---|---|---|---|---|
| Week | Mean | Mean | Mean | Mean | Mean |
| 0 | 0% | 0% | 0% | 0% | 0% |
| 1 | −7% | −4% | −8% | −3% | −7% |
| 2 | −9% | −5% | −13% | −12% | −12% |
| 3 | −14% | −7% | −17% | −20% | −11% |
| 4 | −15% | −10% | −20% | −24% | −14% |
| 5 | −17% | −7% | −21% | −31% | −18% |
| 6 | −22% | −14% | −26% | −30% | −19% |
| 7 | −20% | −12% | −27% | −34% | −20% |
| 8 | −24% | −14% | −28% | −37% | −22% |
| 9 | −24% | −14% | −28% | −38% | −23% |
| 10 | −26% | −15% | −29% | −40% | −23% |
| 11 | −25% | −18% | −25% | −42% | −28% |
| 12 | −28% | −17% | −28% | −44% | −27% |

From this table, it can be seen that the 15 mg daily dose resulted in a reduction of over 40% in the severity of moderate to severe VMS when compared to baseline.
b) Covariate significance

The Table below presents a comparison over all treatment groups.

| Analysis | | |
|---|---|---|
| Timepoint | | |
| (N) | Covariate | p_value |
| 4 | trt1 | 0.0126 |
| 4 | base | 0.6594 |
| 12 | trt1 | 0.0031 |
| 12 | base | 0.1651 |

c) The Table below presents mean relative change from baseline by week and treatment

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.140987 | −0.200215 | −0.081759 |
| 4 | 2.5 | −0.145345 | −0.205600 | −0.085090 |
| 4 | 5 | −0.095556 | −0.159566 | −0.031546 |
| 4 | 10 | −0.201564 | −0.261739 | −0.141388 |
| 4 | 15 | −0.242707 | −0.305276 | −0.180139 |
| 12 | 0 | −0.279744 | −0.368327 | −0.191161 |
| 12 | 2.5 | −0.275038 | −0.365158 | −0.184919 |
| 12 | 5 | −0.164881 | −0.260616 | −0.069145 |
| 12 | 10 | −0.286159 | −0.376158 | −0.196159 |
| 12 | 15 | −0.437476 | −0.531055 | −0.343897 |

d) The Table below presents differences with placebo by week and treatment

| Anal- | | | | | | |
|---|---|---|---|---|---|---|
| ysis | | | | Lower | Upper | |
| Time- | Higher | Lower | LS | 95% | 95% | |
| point | dose | dose | adjusted | confidence | confidence | |
| (N) | (mg) | (mg) | mean | limit | limit | p_value |
| 4 | 2.5 | 0 | −0.004358 | −0.110211 | 0.101495 | 0.9999 |
| 4 | 5.0 | 0 | 0.045431 | −0.063853 | 0.154714 | 0.7006 |
| 4 | 10.0 | 0 | −0.060577 | −0.166020 | 0.044866 | 0.4281 |
| 4 | 15.0 | 0 | −0.101720 | −0.209363 | 0.005922 | 0.0702 |
| 12 | 2.5 | 0 | 0.004705 | −0.153612 | 0.163022 | 1.0000 |
| 12 | 5.0 | 0 | 0.114863 | −0.048585 | 0.278310 | 0.2522 |
| 12 | 10.0 | 0 | −0.006415 | −0.164119 | 0.151289 | 0.9999 |
| 12 | 15.0 | 0 | −0.157732 | −0.318726 | 0.003261 | 0.0568 |

From this Table it can be seen that the 15 mg daily dose generates a near statistically significant difference with placebo at 12 weeks (p-value of 0.0568). The 15 mg daily dose improves the severity of VMS at weeks 4 and 12 compared with placebo, whereas the 10 mg daily dose here again is hardly distinguishable from placebo, especially so at 12 weeks.

c. Hot Flush Weekly Weighted Score
i. Absolute Change (Mean Change From Baseline) in Weekly Weighted Score
a) Week-by-week for each group

| | 2.5 mg | 5 mg | 10 mg | 15 mg | Placebo |
|---|---|---|---|---|---|
| Week | Mean | Mean | Mean | Mean | Mean |
| 0 | 2.4151 | 1.8297 | 2.3773 | 2.219 | 2.0545 |
| 1 | −40.805 | −33.7092 | −31.4591 | −33.1745 | −38.203 |
| 2 | −58.6163 | −49.1319 | −54.0849 | −67.6983 | −64.1272 |
| 3 | −74.8798 | −56.9697 | −73.5189 | −85.1915 | −70.45 |
| 4 | −82.75 | −64.8344 | −87.2673 | −98.8329 | −77.936 |
| 5 | −84.6062 | −71.6181 | −93.4648 | −105.223 | −83.073 |
| 6 | −89.9842 | −88.2337 | −101.646 | −110.268 | −89.6742 |
| 7 | −94.2468 | −88.2989 | −103.024 | −115.263 | −92.9248 |
| 8 | −97.1084 | −89.2039 | −104.835 | −115.656 | −91.6603 |
| 9 | −101.429 | −90.923 | −107.578 | −118.578 | −94.9918 |
| 10 | −100.18 | −91.4798 | −109.514 | −116.853 | −99.4484 |
| 11 | −97.6016 | −95.8365 | −109.532 | −119.365 | −102.771 |
| 12 | −98.5424 | −93.5117 | −111.764 | −121.777 | −104.292 |

b) Covariate significance

The Table below presents a comparison over all treatment groups.

| Analysis | | |
|---|---|---|
| Timepoint | | |
| (N) | Covariate | p_value |
| 4 | trt1 | 0.0128 |
| 4 | base | <.0001 |
| 12 | trt1 | 0.0107 |
| 12 | base | <.0001 |

c) The Table below presents mean change from baseline by week and treatment:

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −78.267781 | −93.531339 | −63.004223 |
| 4 | 2.5 | −75.370737 | −91.064629 | −59.676845 |
| 4 | 5 | −66.878234 | −83.400001 | −50.356467 |
| 4 | 10 | −85.563378 | −101.119727 | −70.007030 |
| 4 | 15 | −106.330686 | −122.645517 | −90.015855 |
| 12 | 0 | −104.682568 | −120.535991 | −88.829145 |
| 12 | 2.5 | −89.857250 | −106.157637 | −73.556864 |
| 12 | 5 | −95.915504 | −113.075759 | −78.755249 |
| 12 | 10 | −109.760136 | −125.917663 | −93.602609 |
| 12 | 15 | −130.594733 | −147.540056 | −113.649411 |

d) The Table below presents differences with placebo by week and treatment

| Anal- | | | | | | |
|---|---|---|---|---|---|---|
| ysis | | | | Lower | Upper | |
| Time- | Higher | Lower | LS | 95% | 95% | |
| point | dose | dose | adjusted | confidence | confidence | p_ |
| (N) | (mg) | (mg) | mean | limit | limit | value |
| 4 | 2.5 | 0 | 2.897044 | −24.495634 | 30.289722 | 0.9970 |
| 4 | 5.0 | 0 | 11.389547 | −16.739876 | 39.518971 | 0.7205 |
| 4 | 10.0 | 0 | −7.295597 | −34.556270 | 19.965075 | 0.9147 |
| 4 | 15.0 | 0 | −28.062905 | −55.994318 | −0.131492 | 0.0485 |
| 12 | 2.5 | 0 | 14.825318 | −13.625957 | 43.276593 | 0.5176 |
| 12 | 5.0 | 0 | 8.767064 | −20.449428 | 37.983557 | 0.8780 |
| 12 | 10.0 | 0 | −5.077568 | −33.391737 | 23.236601 | 0.9785 |
| 12 | 15.0 | 0 | −25.912165 | −54.922995 | 3.098664 | 0.0951 |

ii. Relative Change (% from Baseline) in Weekly Weighted Score
a) Week-by-week for each group

| | 2.5 mg | 5 mg | 10 mg | 15 mg | Placebo |
|---|---|---|---|---|---|
| Week | Mean | Mean | Mean | Mean | Mean |
| 0 | 0% | 0% | 0% | 0% | 0% |
| 1 | −22% | −22% | −20% | −19% | −24% |
| 2 | −32% | −32% | −35% | −43% | −40% |
| 3 | −43% | −37% | −47% | −56% | −43% |
| 4 | −47% | −43% | −54% | −66% | −47% |
| 5 | −48% | −46% | −58% | −70% | −51% |
| 6 | −51% | −56% | −64% | −74% | −55% |
| 7 | −53% | −56% | −64% | −78% | −57% |
| 8 | −55% | −56% | −66% | −78% | −57% |
| 9 | −57% | −58% | −67% | −80% | −58% |
| 10 | −57% | −59% | −68% | −79% | −62% |
| 11 | −56% | −61% | −69% | −80% | −64% |
| 12 | −57% | −60% | −70% | −82% | −65% |

b) Covariate significance

The Table below presents a comparison over all treatment groups.

| Analysis | | |
|---|---|---|
| Timepoint | | |
| (N) | Covariate | p_value |
| 4 | trt1 | 0.0108 |
| 4 | base | 0.4181 |
| 12 | trt1 | 0.0024 |
| 12 | base | 0.0593 |

c) The Table below presents mean relative change from baseline by week and treatment

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.474107 | −0.562044 | −0.386170 |
| 4 | 2.5 | −0.473966 | −0.564382 | −0.383549 |
| 4 | 5 | −0.426177 | −0.521362 | −0.330991 |
| 4 | 10 | −0.541886 | −0.631510 | −0.452262 |
| 4 | 15 | −0.650317 | −0.744310 | −0.556323 |
| 12 | 0 | −0.644566 | −0.728624 | −0.560509 |
| 12 | 2.5 | −0.579457 | −0.665884 | −0.493029 |
| 12 | 5 | −0.599251 | −0.690237 | −0.508264 |
| 12 | 10 | −0.699351 | −0.785021 | −0.613681 |
| 12 | 15 | −0.812214 | −0.902061 | −0.722368 |

d) The Table below presents differences with placebo by week and treatment

| Analysis | | | | Lower | Upper | |
|---|---|---|---|---|---|---|
| Time- | Higher | Lower | LS | 95% | 95% | |
| point | dose | dose | adjusted | confidence | confidence | p_ |
| (N) | (mg) | (mg) | mean | limit | limit | value |
| 4 | 2.5 | 0 | −0.473966 | −0.564382 | −0.383549 | 1.0000 |
| 4 | 5.0 | 0 | −0.426177 | −0.521362 | −0.330991 | 0.8833 |
| 4 | 10.0 | 0 | −0.541886 | −0.631510 | −0.452262 | 0.6745 |
| 4 | 15.0 | 0 | −0.650317 | −0.744310 | −0.556323 | 0.0267 |
| 12 | 2.5 | 0 | −0.579457 | −0.665884 | −0.493029 | 0.6744 |
| 12 | 5.0 | 0 | −0.599251 | −0.690237 | −0.508264 | 0.8871 |
| 12 | 10.0 | 0 | −0.699351 | −0.785021 | −0.613681 | 0.7861 |
| 12 | 15.0 | 0 | −0.812214 | −0.902061 | −0.722368 | 0.0276 |

From this Table it can be seen that the 15 mg daily dose generates a statistically significant difference with placebo at 4 weeks (p-value of 0.0267) and at 12 weeks (p-value of 0.0276). It is particularly striking to observe the low p-values obtained for the 15 mg dose by comparison to the elevated p-values obtained for the 10 mg dose.

### B. Vasomotors Parameters for the 10 mg and 15 mg Groups by Comparison to the Placebo and Inefficient Doses (2.5 mg and 5 mg) Grouped Together

Based on the results observed in section A above, it became apparent that the two lowest doses tested (2.5 mg per day and 5 mg per day) did not show efficacy. A further analysis of the results was thus prepared, where data from these two doses were grouped with the placebo dose and compared to the 10 mg and 15 mg doses.

1. Relative Change (% from Baseline) in Weekly Frequency of Moderate to Severe VMS
a) Week-by-week for each group

| | | | | | Placebo with | |
|---|---|---|---|---|---|---|
| | 10 mg | | 15 mg | | 2.5 and 5 mg | |
| Week | Mean | SD | Mean | SD | Mean | SD |
| 0 | 0% | 0% | 0% | 0% | 0% | 0% |
| 1 | −21% | 29% | −22% | 33% | −24% | 25% |
| 2 | −37% | 37% | −46% | 34% | −35% | 31% |
| 3 | −48% | 39% | −57% | 34% | −42% | 32% |
| 4 | −55% | 35% | −67% | 30% | −47% | 33% |
| 5 | −60% | 33% | −72% | 28% | −50% | 33% |
| 6 | −65% | 33% | −76% | 26% | −56% | 32% |
| 7 | −66% | 34% | −79% | 25% | −58% | 32% |
| 8 | −68% | 35% | −79% | 26% | −58% | 34% |
| 9 | −69% | 36% | −81% | 24% | −60% | 33% |
| 10 | −70% | 35% | −80% | 27% | −61% | 33% |
| 11 | −70% | 34% | −82% | 25% | −63% | 34% |
| 12 | −72% | 33% | −84% | 23% | −63% | 33% |

b) Covariate significance

| Analysis | | |
|---|---|---|
| Timepoint | | |
| (N) | Covariate | p_value |
| 4 | trt1 | 0.0039 |
| 4 | base | 0.3935 |
| 4 | SITEPOOL | 0.3308 |
| 12 | trt1 | 0.0017 |
| 12 | base | 0.1244 |
| 12 | SITEPOOL | 0.0957 |

c) Pairwise comparisons with placebo (including ineffective doses 2.5 mg and 5 mg)

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.46779276 | −0.521579 | −0.414007 |
| 4 | 10 | −0.54424142 | −0.634204 | −0.454279 |
| 4 | 15 | −0.64752092 | −0.741851 | −0.553191 |
| 12 | 0 | −0.61799510 | −0.669162 | −0.566828 |
| 12 | 10 | −0.70075417 | −0.786337 | −0.615172 |
| 12 | 15 | −0.80058459 | −0.890322 | −0.710847 |

d) Differences with placebo (including ineffective doses 2.5 mg and 5 mg)

| Analysis | | | | Lower | Upper | |
|---|---|---|---|---|---|---|
| Time- | Higher | Lower | LS | 95% | 95% | |
| point | dose | dose | adjusted | confidence | confidence | |
| (N) | (mg) | (mg) | mean | limit | limit | p_value |
| 4 | 10.0 | 0 | −0.076449 | −0.193421 | 0.040523 | 0.26148 |
| 4 | 15.0 | 0 | −0.179728 | −0.301777 | −0.057679 | 0.00218 |
| 12 | 10.0 | 0 | −0.082759 | −0.194036 | 0.028518 | 0.17979 |
| 12 | 15.0 | 0 | −0.182589 | −0.298696 | −0.066483 | 0.00099 |

As already mentioned under Section A) above it is particularly striking to observe the low p-values obtained for the 15 mg daily dose by comparison to the elevated p-values obtained for the 10 mg daily dose, demonstrating the unique relief obtained with the 15 mg daily dose.

2. Relative Change (% from Baseline) in Hot Flush Weekly Weighted Score
a) Week-by-week for each group

| | | | | | Placebo with | |
|---|---|---|---|---|---|---|
| | 10 mg | | 15 mg | | 2.5 and 5 mg | |
| Week | Mean | SD | Mean | SD | Mean | SD |
| 0 | 0% | 0% | 0% | 0% | 0% | 0% |
| 1 | −20% | 28% | −22% | 32% | −24% | 25% |
| 2 | −34% | 36% | −45% | 32% | −36% | 30% |
| 3 | −46% | 39% | −56% | 32% | −42% | 31% |
| 4 | −53% | 34% | −66% | 29% | −46% | 32% |
| 5 | −57% | 32% | −70% | 27% | −49% | 33% |
| 6 | −62% | 32% | −72% | 26% | −54% | 32% |
| 7 | −64% | 32% | −75% | 25% | −55% | 31% |
| 8 | −66% | 34% | −76% | 26% | −56% | 32% |
| 9 | −66% | 34% | −78% | 25% | −57% | 31% |
| 10 | −68% | 34% | −77% | 27% | −59% | 32% |
| 11 | −68% | 33% | −79% | 26% | −60% | 33% |
| 12 | −69% | 32% | −81% | 24% | −60% | 32% |

b) Covariate significance

| Analysis | | |
|---|---|---|
| Timepoint | | |
| (N) | Covariate | p_value |
| 4 | trt1 | 0.0037 |
| 4 | base | 0.5294 |
| 4 | SITEPOOL | 0.3783 |
| 12 | trt1 | 0.0015 |
| 12 | base | 0.0770 |
| 12 | SITEPOOL | 0.0487 |

c) Pairwise comparisons with placebo (including ineffective doses 2.5 mg and 5 mg)

| Analysis | Planned | | Lower 95% | Upper 95% |
|---|---|---|---|---|
| Timepoint | treatment | LS adjusted | confidence | confidence |
| (N) | (mg) | mean | limit | limit |
| 4 | 0 | −0.45362100 | −0.506219 | −0.401023 |
| 4 | 10 | −0.51368261 | −0.601866 | −0.425499 |
| 4 | 15 | −0.63242707 | −0.724866 | −0.539989 |
| 12 | 0 | −0.59132448 | −0.640861 | −0.541788 |
| 12 | 10 | −0.67757463 | −0.760625 | −0.594524 |
| 12 | 15 | −0.76832743 | −0.855386 | −0.681269 |

d) Differences with placebo (including ineffective doses 2.5 mg and 5 mg)

| Analysis | | | | Lower | Upper | |
|---|---|---|---|---|---|---|
| Time- | Higher | Lower | LS | 95% | 95% | |
| point | dose | dose | adjusted | confidence | confidence | |
| (N) | (mg) | (mg) | mean | limit | limit | p_value |
| 4 | 10.0 | 0 | −0.060062 | −0.174760 | 0.054636 | 0.41666 |
| 4 | 15.0 | 0 | −0.178806 | −0.298114 | −0.059498 | 0.00178 |
| 12 | 10.0 | 0 | −0.086250 | −0.194272 | 0.021772 | 0.14044 |
| 12 | 15.0 | 0 | −0.177003 | −0.289367 | −0.064639 | 0.00097 |

As already mentioned under Section A) above, it can be seen that the 15 mg daily dose generates a statistically significant difference with placebo at 4 weeks (p-value of 0.00178) and at 12 weeks (p-value of 0.00097).

Again, it is particularly striking to observe the low p-values obtained for the 15 mg dose by comparison to the elevated p-values obtained for the 10 mg dose.

### C. Menopause Rating Scale

The Menopause Rating Scale (MRS) is a health-related quality of life scale allowing the measure of severity of age-/menopause-related complaints by rating a profile of symptoms (Heinemann et al., 2003, “International versions of the Menopause Rating Scale (MRS)” Health Qual Life Outcomes 1:28; Heinemann et al., 2004, “The Menopause Rating Scale (MRS) scale: A methodological review”. Health Qual Life Outcomes 2:45; Heinemann et al., 2004, “The Menopause Rating Scale (MRS) as outcome measure for hormone treatment? A validation study”. Health Qual Life Outcomes 2:67).

The score increases point by point with increasing severity of subjectively perceived complaints in each one of 11 items (severity expressed in 0 to 4 points in each item). By checking these 5 possible boxes of “severity” for each of the items in the questionnaire, the respondent provides her personal perception. The total MRS score ranges between 0 (asymptomatic) to 44 (highest degree of complaints). The minimal/maximal scores vary between three dimensions depending on the number of complaints allocated to the respective dimension of symptoms (Heinemann et al., 2003, Health Qual Life Outcomes 1:28):
1. psychological symptoms: 0 to 16 scoring points (4 symptoms: depressed, irritable, anxious, exhausted);
2. somato-vegetative symptoms: 0 to 16 points (4 symptoms: sweating/flush, cardiac complaints, sleeping disorders, joint & muscle complaints);
3. urogenital symptoms: 0 to 12 points (3 symptoms: sexual problems, urinary complaints, vaginal dryness).

### Total MRS Score

| | 2.5 mg | | 5 mg | | 10 mg | | 15 mg | | Placebo | |
|---|---|---|---|---|---|---|---|---|---|---|
| Week | Mean | SD | Mean | SD | Mean | SD | Mean | SD | Mean | SD |
| Baseline | 16.5 | 7.2 | 16.5 | 7.3 | 17.6 | 7.6 | 16.4 | 8.1 | 18.2 | 8.9 |
| Week 4 | 10.3 | 5.6 | 10.5 | 6.7 | 11.7 | 6.8 | 8.7 | 6.2 | 12.8 | 8.0 |
| Week 12 | 9.5 | 6.7 | 11.0 | 7.7 | 9.7 | 6.9 | 8.1 | 5.8 | 11.4 | 7.8 |

The Menopause Rating Scale (MRS) points to an overall improvement in quality of life, with the strongest effect for the 15 mg dose. At this dose, a statistically significant effect was observed by comparison with placebo at week 4, with a p-value of 0.0113 and a near statistically significant effect was observed at week 12, with a p-value of 0.0694.

### D. Genito-urinary symptoms (GSM)

Change from baseline to week 12 in the following GSM symptoms (VVA subject self-assessment) were recorded:
a) Vaginal dryness (sensation of dryness or burning in the vagina; none=0 mild=1, moderate=2 or severe=3):

| Dose E4 | Baseline | Week 12 | p-value vs |
|---|---|---|---|
| (mg) | (mean ± SD) | (mean ± SD) | placebo |
| 2.5 | 1 ± 0.90 | 0.5 ± 0.77 | 0.3345 |
| 5 | 1.3 ± 0.94 | 0.7 ± 0.86 | 0.1202 |
| 10 | 1 ± 0.93 | 0.5 ± 0.75 | 0.0798 |
| 15 | 1.1 ± 1.04 | 0.5 ± 0.68 | 0.0291* |
| Placebo | 1.3 ± 1.10 | 0.9 ± 1.02 | |

*p < 0.05 vs placebo at week 12.

b) Vaginal and/or vulvar irritation/itching (sensation of abnormal irritation or sensitive condition in the vagina; none=0, mild=1, moderate=2 or severe=3):

| Dose E4 | Baseline | Week 12 | p-value vs |
|---|---|---|---|
| (mg) | (mean ± SD) | (mean ± SD) | placebo |
| 2.5 | 0.7 ± 0.96 | 0.3 ± 0.60 | 0.1717 |
| 5 | 0.6 ± 0.90 | 0.4 ± 0.65 | 0.9618 |
| 10 | 0.7 ± 0.87 | 0.3 ± 0.64 | 0.2487 |
| 15 | 0.5 ± 0.85 | 0.4 ± 0.70 | 0.931 |
| Placebo | 0.8 ± 0.92 | 0.5 ± 0.77 | |

c) Dysuria (sensation of pain or difficulty in urinating; none-0, mild=1, moderate=2 or severe=3):

| Dose E4 | Baseline | Week 12 | p-value vs |
|---|---|---|---|
| (mg) | (mean ± SD) | (mean ± SD) | placebo |
| 2.5 | 0.2 ± 0.56 | 0 ± 0.19 | 0.2942 |
| 5 | 0.2 ± 0.56 | 0 ± 0.20 | 0.3488 |
| 10 | 0.2 ± 0.58 | 0.1 ± 0.23 | 0.3386 |
| 15 | 0.3 ± 0.58 | 0.3 ± 0.61 | 0.643 |
| Placebo | 0.2 ± 0.60 | 0.3 ± 0.55 | |

d) Vaginal pain associated with sexual activity (sensation of pain with sexual intercourse none=0, mild=1, moderate=2 or severe=3):

| Dose E4 | Baseline | Week 12 | p-value vs |
|---|---|---|---|
| (mg) | (mean ± SD) | (mean ± SD) | placebo |
| 2.5 | 0.6 ± 0.77 | 0.3 ± 0.73 | 0.0763 |
| 5 | 1 ± 1.07 | 0.5 ± 0.78 | 0.0246* |
| 10 | 0.6 ± 0.77 | 0.2 ± 0.36 | 0.0004** |
| 15 | 0.7 ± 0.90 | 0.3 ± 0.54 | 0.0006** |
| Placebo | 1 ± 1.14 | 0.7 ± 1.01 | |

*p < 0.05 vs piacebo at week 12;
**p < 0.001 vs placebo at week 12.

e) Vaginal bleeding associated with sexual activity (loss of blood with sexual intercourse; presence=1 vs. absence=0):

| Dose | Baseline | | Week 12 | | |
|---|---|---|---|---|---|
| E4 | Presence | Absence | Presence | Absence | p-value vs |
| (mg) | (%) | (%) | (%) | (%) | placebo |
| 2.5 | 0 | 100 | 0 | 100 | 0.9958 |
| 5 | 6.4 | 91.5^{#} | 0 | 100 | 0.903 |
| 10 | 0 | 100 | 0 | 100 | 0.9955 |
| 15 | 0 | 97.9^{#} | 4.2 | 95.8 | 0.9308 |
| Placebo | 3.6 | 92.7^{#} | 3.6 | 96.4 | |

^{#}Some patients had no sexual activity

The evolution of the VVA symptoms points to an overall improvement, with the strongest effect for the 15 mg daily dose. For vaginal pain associated with sexual activity, significant differences with placebo are observed with the doses of 5, 10 and 15 mg daily with p-values of 0.0246, 0.0004 and 0.0006, respectively. Vaginal dryness, however, which is generally considered as the most bothersome symptom, is only significantly improved by the 15 mg daily dose, with a p-value of 0.0291.

### E. Measurements Related to Treatment Side Effects

1. Number of Patients with Biopsies

| Treatment Group | Placebo | 2.5 mg | 5 mg | 10 mg | 15 mg |
|---|---|---|---|---|---|
| Number of patients | 4 | 4 | 5 | 11 | 9 |
| with biopsies | | | | | |

### 2. Adverse Events (AEs)

| Treatment Group | Placebo | 2.5 mg | 5 mg | 10 mg | 15 mg |
|---|---|---|---|---|---|
| Count of Treatment | 71 | 61 | 63 | 95 | 82 |
| Emergent | | | | | |
| AEs (TEAEs) | | | | | |
| Percentage of patients | 9.1 | 7.7 | 10.6 | 7.4 | 6.1 |
| with severe TEAE | | | | | |
| Percentage of | 3.6 | 1.9 | 6.4 | 5.6 | 4.1 |
| patients with TEAE | | | | | |
| leading to | | | | | |
| Study Discontinuation | | | | | |

It can be seen from the Table above that the patients in the 15 mg group present less TEAEs than patients in the 10 mg group. In the 10 mg group of patients who had AEs, the average was 3.2 AEs per patient. By comparison, in the 15 mg group, patients who had AEs had on average 2.6 AEs. Globally, those data show that the 15 mg daily dose provides a significant relief of VMS without generating additional AEs for the patients. In addition, there were less requirements for biopsies in the 15 mg per day group than in the 10 mg per day group.

This is confirmed by the following statistical analysis. Using a Poisson regression model with a random effect for the patient and treatment group as a covariate to model the count of TEAEs in the different treatment groups, it can be shown that there is no statistical difference between the treatment groups (p value of 0.099). Second, a chi-square test was used to assess if the prevalence of patients reporting TEAEs in each treatment group was similar. No statistical difference was found between the treatment groups (p value of 0.575).

### 3. Patients Leaving the Study

| Treatment Group | Placebo | 2.5 mg | 5 mg | 10 mg | 15 mg |
|---|---|---|---|---|---|
| Number of patients | 14 | 9 | 11 | 15 | 8 |
| leaving the study | | | | | |

### F. Measurements Performed at 15 mg and 20 mg Daily Doses

In order to better assess the potential for increasing the daily dose beyond the minimum effective dose of 15 mg daily, a number of parameters were followed in a study where estetrol was administered at the increased dose of 20 mg per day.

1. Triglycerides Level (mmol/L)

| | | | End Of Treatment * | |
|---|---|---|---|---|
| | | | Actual | Actual Change |
| Treatment | Parameter | Baseline | Value | from Baseline |
| E4 15 mg | Mean | 1.32 | 1.61 | 0.20 |
| | SD | 0.64 | 0.94 | 0.82 |
| | Median | 1.21 | 1.40 | 0.15 |
| E4 20 mg | Mean | 1.43 | 1.53 | 0.10 |
| | SD | 0.47 | 0.63 | 0.44 |
| | Median | 1.40 | 1.40 | 0.10 |

* End Of Treatment was after 28 days for the 20 mg dose and after 12 weeks for the 15 mg dose.

2. Glucose Level (mmol/L)

| | | | End Of Treatment * | |
|---|---|---|---|---|
| | | | Actual | Actual Change |
| Treatment | Parameter | Baseline | Value | from Baseline |
| E4 15 mg | Mean | 4.77 | 4.79 | 0.02 |
| | SD | 0.37 | 0.58 | 0.56 |
| | Median | 4.72 | 4.77 | 0.05 |
| E4 20 mg | Mean | 5.53 | 5.44 | −0.10 |
| | SD | 0.54 | 0.50 | 0.22 |
| | Median | 5.60 | 5.60 | −0.15 |

* End Of Treatment was after 28 days for the 20 mg dose and after 12 weeks for the 15 mg dose.

3. Cholesterol Level (mmol/L)

| | | | End Of Treatment * | |
|---|---|---|---|---|
| | | | Actual | Actual Change |
| Treatment | Parameter | Baseline | Value | from Baseline |
| E4 15 mg | Mean | 5.51 | 5.63 | 0.12 |
| | SD | 0.83 | 0.97 | 0.73 |
| | Median | 5.62 | 5.71 | 0.08 |
| E4 20 mg | Mean | 6.27 | 6.09 | −0.18 |
| | SD | 0.90 | 0.96 | 0.65 |
| | Median | 6.25 | 6.10 | −0.20 |

* End Of Treatment was after 28 days for the 20 mg dose and after 12 weeks for the 15 mg dose.

10 4. HDL-Cholesterol Level (mmol/L)

| | | | End Of Treatment * | |
|---|---|---|---|---|
| | | | Actual | Actual Change |
| Treatment | Parameter | Baseline | Value | from Baseline |
| E4 15 mg | Mean | 1.73 | 1.89 | 0.16 |
| | SD | 0.46 | 0.41 | 0.24 |
| | Median | 1.66 | 1.81 | 0.17 |
| E4 20 mg | Mean | 1.68 | 1.97 | 0.29 |
| | SD | 0.37 | 0.36 | 0.14 |
| | Median | 1.60 | 1.90 | 0.35 |

* End Of Treatment was after 28 days for the 20 mg dose and after 12 weeks for the 15 mg dose.

5. LDL-Cholesterol Level (mmol/L)

| | | | End Of Treatment * | |
|---|---|---|---|---|
| | | | Actual | Actual Change |
| Treatment | Parameter | Baseline | Value | from Baseline |
| E4 15 mg | Mean | 3.05 | 3.14 | 0.09 |
| | SD | 0.85 | 0.95 | 0.45 |
| | Median | 3.22 | 3.25 | 0.10 |
| E4 20 mg | Mean | 3.94 | 3.43 | −0.51 |
| | SD | 0.98 | 1.05 | 0.56 |
| | Median | 4.00 | 3.45 | −0.65 |

* End Of Treatment was after 28 days for the 20 mg dose and after 12 weeks for the 15 mg dose.

From the above 5 Tables it can be observed that these lipid parameters and glucose level do not behave significantly differently when a 20 mg daily dose is used in place of a 15 mg daily dose.

6. C-Terminal Telopeptide (CTX-1) (ng/L)

| | | | End Of Treatment * | |
|---|---|---|---|---|
| | | | Actual | Actual Change |
| Treatment | Parameter | Baseline | Value | from Baseline |
| E4 15 mg | Mean | 416.0 | 320.9 | −95.0 |
| | SD | 286.16 | 359.65 | 442.18 |
| | Median | 339.0 | 197.5 | −142.5 |
| E4 20 mg | Mean | 422.50 | 274.9 | −147.60 |
| | SD | 134.8 | 95.3 | 70.6 |
| | Median | 416.5 | 267.50 | −138.0 |

* End Of Treatment was after 28 days for the 20 mg dose and after 12 weeks for the 15 mg dose.

CTX-1 is a specific marker of bone resorption.

In the Table above it can been seen that the 15 mg daily dose leads to a small decrease of bone resorption and this effect is more pronounced with the 20 mg daily dose already after 28 days of treatment.

### G. Measurements Performed at 15 mg and 30 mg Daily Doses

The following treatments were administered to healthy women (between 15-50 years inclusive) according to the randomization code.
Placebo (n=16);
Group 15 mg: a single oral dose of 15 mg Estetrol/3 mg Drospirenone (n=10) followed, after a washout of 14 days, by multiple oral doses of 15 mg Estetrol/3 mg Drospirenone (n=10) once daily for 14 days;
Group 30 mg: a single oral dose of 30 mg Estetrol/6 mg Drospirenone (n=10), followed, after a washout of 14 days, by multiple oral doses of 30 mg Estetrol/6 mg Drospirenone (n=10) once daily for 14 days.

Adverse events were recorded from first admission until completion of the follow-up visit (between 37 to 42 days after first day of treatment).

| Treatment Emergent AEs (TEAEs) by | | | |
|---|---|---|---|
| relationship to study drug | Placebo | 15 mg | 30 mg |
| Total | 75% | 80% | 70% |
| Related | 31% | 50% | 50% |
| Unrelated | 75% | 50% | 50% |

% = number of subjects reporting one or more AE as percentage of the total number of subjects in the corresponding treatment group

Overall, single dose administration and 14-day once daily administration of oral Estetrol/Drospirenone doses in the range of 15 mg Estetrol/3 mg Drospirenone to 30 mg Estetrol/6 mg Drospirenone were safe and well-tolerated by the healthy female subjects in this study. With increasing single and multiple Estetrol/Drospirenone doses (dose was doubled), no increase in either percentage of subjects reporting TEAEs or the number of TEAEs was observed

It is thus reasonable to envision a hormone replacement therapy for alleviating menopause-associated symptoms which uses a daily dose of estetrol of between 15 mg, the minimal effective dose, and 20 mg, or even 25 mg which will allow a better benefit-to-risk profile to be obtained. Increasing the estetrol dose beyond the minimal effective dose of 15 mg per day will indeed offer even better efficacy in VMS relief and in parameters such as bone resorption, while maintaining an excellent safety profile (including, but not limited to, Adverse Events, as presented under Sections E)2) and G) above, and particularly lipid parameters and glucose level presented under Section F)1) to 5) above). Increasing the estetrol dose beyond the minimal effective dose of 15 mg per day will also permit a faster onset of relief to be obtained when patients initiate hormone replacement therapy.

1-15. (canceled)

16. A method of treating moderate to severe vasomotor symptoms (VMS) associated with menopause, wherein said method comprises orally administering an estetrol component at a daily amount equivalent to about 20 mg of estetrol to a subject in need thereof.

17. The method according to claim 16, wherein the administration of said estetrol component to the subject does not modify one or more haemostatic parameters of the subject.

18. The method according to claim 16, wherein said estetrol component is an estetrol hydrate.

19. The method according to claim 16, wherein said estetrol component is estetrol monohydrate.

20. The method according to claim 16, wherein said menopause is characterized by thermo-regulatory disturbances in the subject.

21. The method according to claim 16, wherein the moderate to severe VMS comprise episodes of profuse heat accompanied by sweating and flushing, experienced around the head, neck, chest, and/or upper back.

22. The method according to claim 21, wherein the subject has at least 7 moderate to severe VMS episodes per day or at least 50 moderate to severe VMS episodes per week.

23. The method according to claim 21, wherein a VMS episode is considered moderate when the subject experiences a sensation of heat with sweating and is able to continue activity.

24. The method according to claim 21, wherein a VMS episode is considered severe when the subject experiences a sensation of heat with sweating which causes cessation of activity.

25. The method according to claim 16, wherein endometrial thickness is not altered by the administration of said estetrol component to the subject.

26. The method according to claim 16, further comprising administering a progestogenic component to the subject.

27. The method according to claim 16, wherein the estetrol component is comprised in a dosage unit.

28. The method according to claim 27, wherein the dosage unit is an oral dosage unit.

29. The method according to claim 28, wherein the oral dosage unit is a tablet.

30. The method according to claim 27, wherein the dosage unit is a daily dosage unit.

31. The method according to claim 16, wherein the estetrol component is administered daily for at least 1, at least 2, at least 4, at least 6, or at least 12 weeks.

32. The method according to claim 16, wherein the subject is a postmenopausal woman.

33. The method according to claim 32. wherein the postmenopausal woman has an estradiol concentration of less than 20 pg/ml and a follicle-stimulating hormone concentration of more than 40 IU/L.

34. The method according to claim 16, wherein the subject is a non- hysterectomieed woman.
